(19) United States
(12) Patent Application Publication
(10) Pub. No.: US 2021/0023292 A1
(43) Pub. Date: Jan. 28, 2021

(54) EXTRACORPOREAL MEMBRANE OXYGENATION APPARATUSES AND METHODS OF THEIR PREPARATION AND USE

(71) Applicant: FREEFLOW MEDICAL DEVICES LLC, Lancaster, PA (US)

(72) Inventors: Saibal BANDYOPADHYAY, Lancaster, PA (US); Andrew K. JONES, Lancaster, PA (US); Daniel C. LESLIE, Roslindale, MA (US); Matthew J. STERNER, Lancaster, PA (US)

(21) Appl. No.: 16/919,971

(22) Filed: Jul. 2, 2020

Related U.S. Application Data
(63) Continuation of application No. PCT/US2019/012580, filed on Jan. 7, 2019.
(60) Provisional application No. 62/614,295, filed on Jan. 5, 2018.

(51) Int. Cl. A61M 1/36; A61M 1/16; A61M 1/26

(52) U.S. Cl. CPC A61M 1/3673; A61M 1/1698; A61M 2202/0225; A61M 1/267; A61M 2202/0208; A61M 1/1621

(57) ABSTRACT

The present disclosure provides extracorporeal membrane oxygenation (ECMO) apparatuses treated to comprise lubricating liquid surfaces, hydrophobic or omniphobic surfaces, and/or fluid and solid repellant slippery surfaces that do not stimulate clotting and thrombus formation to the same degree as do the untreated surfaces. The apparatuses permit ECMO procedures to be conducted using less anticoagulant and/or antiplatelet medications than is possible with untreated apparatuses. Also described are methods of using the treated apparatuses to accomplish ECMO procedures on subjects (e.g., human patients) and upon perfused organs.

### STATEMENT REGARDING FEDERALLY SPONSORED RESEARCH OR DEVELOPMENT

This invention was made with government support under CRADA MRMC Control No. W81XWH-17-0135 awarded by US Army Institute of Surgical Research. The government has certain rights in the invention.

### BACKGROUND

Extracorporeal membrane oxygenation (ECMO) apparatuses have been principally developed to support the exchange of carbon dioxide for oxygen in the blood of mammalian subjects (e.g., canine, bovine, caprine, hominid including the great apes, primate, murine, ovine, porcine, or human subjects) where their cardio/pulmonary systems is/are insufficient. ECMO systems find use, for example, in the support of subjects or patients whose cardio-pulmonary system has been subject to acute trauma or temporarily stopped to conduct medical procedures, or for patients who suffer from Acute Respiratory Distress Syndrome (ARDS). In some instances, ECMO may be a lifesaving therapeutic option for acute respiratory failure in both infants and adults.

Most, if not all, oxygenators developed to date may be broadly classified as either bubble-type oxygenators or membrane-type oxygenators. The membrane-type oxygenators may be divided into laminate, coil, and hollow fiber types. In addition, membrane-type oxygenators may employ membranes in the form of flat sheets or sheets that are folded (plaque-type) to increase the surface area of the membrane housed in a small exchange chamber. Membrane-type oxygenators are generally considered superior to bubble-type oxygenators as their operation produces less blood damage, such as hemolysis, protein denaturation, and blood coagulation, as compared with bubble-type oxygenators. Still, operation of membrane-type ECMO apparatuses results in blood damage and activation of clotting.

### SUMMARY

Described herein is a method of preparing/treating extracorporeal membrane oxygenator (ECMO) apparatuses (devices) to limit damage caused to blood during their use and limit the colonization by bacteria (e.g., Staphylococcus epidermidis) and subsequent biofilm growth. The treated devices permit the reduction or elimination of anticoagulants (e.g., heparin, warfarin, rivaroxaban, dabigatran, apixaban, edoxaban, enoxaparin, and fondaparinux) and/or antiplatelet medications (e.g., clopidogrel, ticagrelor, prasugrel, dipyridamole, dipyridamole/aspirin, ticlodipine, and eptfibatide) normally required during the use of ECMO devices to support a patient. For example, the dose of anticoagulant (e.g., heparin) required to maintain an activated partial thromboplastin time (APTT or PPT, historically known as the Kaolin cephalin clotting Time or KccT) of 50-70 seconds may be reduced, relative to an ECMO device that is otherwise the same but does not have one or more surfaces that contact blood treated as described herein below. In an embodiment, the use of an ECMO apparatus with all or part of its surfaces that are exposed to blood treated as described below to support a subject (e.g., human or other mammalian patient) reduces the dose of anticoagulant (e.g., heparin) required to maintain an APTT of 50-70 seconds to less than 60%, 50%, 40%, 30%, 20%, 10%, or 5% of the heparin dose required when an untreated, but otherwise identical, ECMO apparatus is employed. Indeed, it is possible to completely omit the need for anticoagulants such as heparin when, for example, the ECMO apparatus incorporates a fluid and solid repellant slippery surface and/or a lubricating liquid surface (e.g., formed on a fluoropolymer and/or perfluoropolymer) on all or part of the apparatus that contacts the blood recirculated to the subject during ECMO. For example, if the dose of heparin currently required to maintain an APTT of 50-70 seconds (following any initial bolus) is about 12 units/kg/hr, it may be reduced to less than about 7.2, less than about 6, less than about 4.8, less than about 3.6, less than about 2.4, or less than about 1.2 units/kg/hr if the ECMO apparatus is treated in accordance with the subject invention. Such reductions in the required amounts of a direct univalent (e.g., Argatroban) or bivalent (e.g., huridin) factor IIa inhibitor or a direct factor Xa inhibitor can also be obtained.

In an embodiment, the disclosure sets forth methods of treating/preparing an ECMO apparatus such that it comprises a fluid and solid repellant slippery surface on all or part of the surfaces which will contact blood recirculated to a subject. Such methods may comprise:
exposing all or part of the apparatus to conditions that produce surface oxygen species capable of reacting with a reactive head group of an anchoring layer molecule and a crosslinking agent which is optionally present (e.g., hydroxyl groups), wherein the anchoring layer molecule comprises the reactive head group and one or more alkyl, fluoroalkyl, or perfluoroalkyl groups, each of which is selected independently; contacting the anchoring layer molecule with the surface oxygen species to form a covalent bond between the head group of the anchoring layer molecule and the surface oxygen species, thereby forming a surface with covalently bound anchoring layer molecules;
removing unreacted anchoring layer molecules; and
contacting at least the part of the apparatus having covalently bound anchoring layer molecules with a lubricating liquid to form a fluid and solid repellant slippery surface at the locations where covalently bound anchoring layer molecules are present.

After treatment with the lubricating liquid, any excess lubricating liquid may be drained from the apparatus, allowed to evaporate or carried away by a stream of gas passed over the treated surfaces. In one such embodiment, at least the surface of the membrane that will contact blood during use of the apparatus is treated.

In an embodiment, the disclosure sets forth methods of treating/preparing an ECMO apparatus such that it comprises a hydrophobic or omniphobic surface on all or part of the surfaces which will contact blood that is recirculated to a subject. Such methods comprise:
exposing all or part of the apparatus to conditions that produce surface oxygen species capable of reacting with a reactive head group of an anchoring layer molecule and a crosslinking agent which is optionally present (e.g., hydroxyl groups), wherein the anchoring layer molecule comprises the reactive head group and one or more alkyl, fluoroalkyl, or perfluoroalkyl groups, each of which is selected independently; contacting the anchoring layer molecule with the surface oxygen species to form a covalent bond between the head group of the anchoring layer molecules and the surface oxygen species, thereby forming a surface with covalently bound anchoring layer molecules; and
removing unreacted anchoring layer molecules.

The resulting surface may have a contact angle with water that is greater than 90°, 100°, 110°, 120°, 130°, 140°, or 160° (e.g., from about 90° to about 120°, about 120° to about 140°, about 140° to about 160°, or about 160° to about)175° measure at 22° C. using a goniometer. Where omniphobic surfaces are desired the anchoring layer molecule comprises one or more fluoroalkyl and/or perfluoroalkyl groups.

In an embodiment, the disclosure sets forth methods of treating/preparing an ECMO apparatus such that all or part of the surfaces which will contact blood that is circulated through the apparatus (e.g., recirculated to a subject or perfused organ) are treated with a lubricating liquid to form a lubricating liquid surface. Such methods comprise contacting at least the part of the apparatus that will be brought into contact with a subject's blood with a lubricating liquid to form a liquid treated surface. After treatment with the lubricating liquid, any excess lubricating liquid may be drained from the apparatus, allowed to evaporate or carried away by a stream of gas passed over the treated surfaces. In one such embodiment, at least the surface of the membrane that will contact blood during use of the apparatus is treated with the lubricating liquid.

Any of the foregoing methods may include a step of cleaning an apparatus prior to any of the treatment steps recited above. For example, cleaning may be used to remove material such as human or bovine serum albumin (HSA or BSA) and/or heparin present on the surface of all or part of the apparatus (e.g., a commercially available apparatus) prior to treating the apparatus with oxygen plasma. In addition, surfaces other than those that contact blood that recirculates to the subject receiving ECMO treatment (e.g., the surface of the membrane that contacts the oxygen source (e.g., oxygen gas or air)) may be subjected to any of the above-mentioned treatments.

The treatments described herein reduce the complications resulting from activation of pathways that lead to blood clotting and, accordingly, the amount of material deposited from blood (clots, fibrin, platelets, etc.) onto the membranes during operation of the ECMO device, relative to the amount observed on an untreated device that is otherwise identical. The amount of thrombus formation may be measured by the fraction (percent) of area covered by thrombi. The amount of deposited material attached to the membranes may be observed directly or by electron microscopy if it is not directly observable by the unaided eye. See, e.g., Beely, et al., ASAIO J. 62(5):525-532 (2016). In an embodiment, the amount of material deposited on the surfaces of an ECMO apparatus treated as described herein is 50%, 60%, 70%, 80%, 90%, or 95% less than the amount of material deposited on an untreated but otherwise identical apparatus operated on a mammalian subject (e.g., a human, chimpanzee, or pig) under identical conditions, with the amount of material deposited being based on the amount of surface area over which material (e.g., coagulated blood) is attached. In another embodiment, the amount of material attached to treated and untreated ECMO apparatuses may be determined by its mass. The mass of material deposited on a treated ECMO apparatus is 50%, 60%, 70%, 80%, 90%, or 95% less than the amount of material deposited on an untreated but otherwise identical apparatus operated on a mammalian subject (e.g., a human, chimpanzee, or pig) under identical conditions. For mass determinations the initial weight of the apparatus and final weight of the apparatus (after rinsing with, for example, normal saline, to remove non-adherent material and drying to constant weight) is used for the comparison.

### Definitions

Extracorporeal membrane oxygenation (ECMO) as used herein is a treatment that uses a pump to circulate blood through an artificial lung (extracorporeal oxygenator) and back into the bloodstream of a patient or subject. An extracorporeal oxygenator, or an oxygenator, as used herein is an apparatus used for ECMO that is capable of exchanging oxygen and carbon dioxide in the blood of a patient or subject outside of the patient or subject's body.

Extracorporeal membrane oxygenator as used herein is an oxygenator that uses a membrane that permits the exchange of oxygen and carbon dioxide in the blood of a patient or subject, where the membrane separates the blood from the source of oxygen, which also acts as the carbon dioxide sink (removal means). The membrane(s) of an ECMO apparatus may have a variety of arrangements including laminate, coil, and hollow fiber arrangements.

For the purposes of this disclosure, a hydrophobic material or surface is one that results in a water droplet forming a static surface contact angle exceeding about 90° at 22° C. at one atmosphere in air measured using a goniometer (e.g., Attension Model Theta goniometer, available from BIOLIN SCIENTIFIC, formerly KSV Instruments, Stockholm, Sweden).

For the purposes of this disclosure, an oleophobic material or surface is one that results in a dodecane droplet forming a static surface contact angle exceeding about 90° at 22° C. at one atmosphere in air measured using a goniometer (e.g., Attension Model Theta goniometer, formerly KSV Instruments, available from BIOLIN SCIENTIFIC, Stockholm, Sweden).

For the purposes of this disclosure, a surface that is omniphobic is both hydrophobic and oleophobic.

As used herein with respect to molecules, “fluorinated” means molecules having fluorine in place of hydrogen. Fluorinated molecules include perfluorinated molecules where all hydrogens have been substituted with a fluorine.

Fluorinated liquids as used herein refer to chemical compositions that are liquid at 22° C. and one atmosphere of pressure comprised of, consisting essentially of, or consisting of hydrocarbons (e.g., alkanes), or molecules having a hydrocarbon moiety (e.g., an alkyl group), in which one or more hydrogen atoms bound to a carbon atom have been replaced by a fluorine atom. Fluorinated liquids include perfluorinated liquids where each hydrogen atom has been replaced by a fluorine atom. The term “fluorinated liquid” is understood to include compositions comprising one or more fluorinated liquids and/or perfluorinated liquids unless stated otherwise.

Where “fluorinated liquid and/or a perfluorinated liquid” is recited it is intended to emphasize the fact fluorinated liquids, perfluorinated liquids, and mixes of fluorinated and perfluorinated liquids can be used.

Fluoropolymers as used herein refer to chemical compositions comprised of, consisting essentially of, or consisting of polymers having a hydrocarbon moiety (e.g., an alkyl group), in which one or more hydrogen atoms bound to a carbon atom have been replaced by a fluorine atom. Fluoropolymers include perfluoropolymers in which each hydrogen atom has been replaced by a fluorine atom. The term “fluoropolymer” is understood to include compositions comprising one or more fluoropolymers and/or perfluoropolymers unless stated otherwise.

As used herein a unit of heparin is equal to 0.002 mg of pure heparin.

### DETAILED DESCRIPTION

### I. ECMO and the Treatment of ECMO Apparatuses

ECMO as a treatment is limited by the damage caused to blood components during passage through the device that can lead to coagulation of blood and thrombosis in the device and in the subject receiving treatment. Damage can be limited by the use of anticoagulants such as heparin and anti-platelet medications such as aspirin; however, this complicates medical treatment and requires monitoring of the subject's status. At their core ECMO units have a membrane oxygenator that is comprised of a housing having a chamber divided into a first and second portion by the gas exchange membrane(s) (oxygenator membrane(s)), with the first portion having a fluid inlet and outlet to permit blood to flow through the first portion and a liquid/gas inlet and outlet to permit an oxygenation media (e.g., gaseous oxygen) to pass through the second portion of the chamber. The apparatus may further contain other components including a pump (e.g., a centrifuge pump), a reservoir, an injection and/or sample port, and tubing (e.g., blood circuit tubing permitting blood to flow from a subject through the oxygenator and other system components and be returned to the subject).

Treatment of ECMO apparatuses to prevent the activation of blood components that lead to thrombosis/coagulation of blood can reduce or eliminate the amount of anticoagulants and/or antiplatelet medications that have to be administered to patients; however, the treatments to the apparatus must not compromise the structural integrity and functionality (e.g., the ability to exchange oxygen and carbon dioxide) of the device, and particularly the membrane that serves to exchange carbon dioxide and oxygen. Membranes employed in ECMO apparatus are generally thin and microporous, having a thickness on the order of 30-50 microns, yet must be able to withstand the pressure associated with pumping blood through the apparatus. Any chemical modification of the membranes, such as exposure to plasma, that alters the membrane has the potential to damage the membrane making it structurally unacceptable (e.g., brittle) or chemically unacceptable (e.g., incompatible with oxygen or carbon dioxide exchange). Moreover, the use of lubricating liquids on such membranes offers the potential that oxygen and/or carbon dioxide transfer efficiency could be lost. The methods described herein may be used to coat/treat the portions of an ECMO apparatus that contact a subject's blood during use, including common gas permeable membranes (including those recited below) used in ECMO apparatus, to form i) a fluid and solid repellant slippery surface, ii) a hydrophobic/omniphobic surface, or iii) a lubricating liquid surface/coating that covers all or part of an apparatus' surface. Not only do the coated/treated surfaces of the ECMO apparatus resist inducing clotting of blood contacted with the surfaces, but they also resist fouling caused by adsorption of plasma proteins (which can lead to the activation of the coagulation cascade, formation of trombin, platelet adhesion and formation of thrombi), colonization and formation of biofilms by bacteria such as S. epidermidis and S. aureus, and by yeast such as Candida. In addition, ECMO apparatus gas exchange membrane(s) are not mechanically damaged/altered by the treatments such that they are capable of being used in standard ECMO apparatus. Furthermore, the oxygen and/or carbon dioxide exchange efficiency (e.g., rate) is greater than or substantially unaltered (e.g., is greater than 70%, 80%, 90%, 95%, or equal to) relative to an otherwise identical but untreated membrane.

ECMO apparatuses that may be treated include laminate, coil, and hollow fiber types, each of which comprises an internal chamber and utilizes one or more membranes (oxygenator/gas exchange membranes) to separate blood in a first part of the apparatus' internal chamber from the gas oxygen/CO₂) exchange medium in a second part of the apparatus' internal chamber, and may provide other functions including, but not limited to, temperature control (the apparatus provides one, two, or more temperature control surfaces).

The treatment methods, described herein below, are gentle and are physically and chemically compatible with the use of ECMO devices employing gas exchange membranes in a variety of configurations including hollow fiber(s), spiral, plaque, and/or flat-sheet(s) having diverse compositions. In an embodiment the membranes are microporous (e.g., microporous hollow fibers), such as microporous perfluoropolymer oxygenator membranes with pore sizes less than, for example 1,000 nanometers (nm) (less than 500 nm, 250 nm, 100 nm, 50 nm, 20 nm, 10 nm, 5 nm, 3 nm, 2 nm, or 1 nm). Hollow fibers typically have a 1-10, 10-50, 50-100, 100-200, 200-300 micron lumen diameter with cross sectional lumen shapes varying from circular/elliptical to polygonal (the number of sides varying from 3, 4, 5, 6, 7, 8, 9 to 10) and may have the internal lumen treated for contact with blood. Some membranes compatible with the treatments methods described herein are those comprised of one or more polymers such as one or more, two or more, or three or more of poly-4-methyl pentene, propylene, FEP (fluorinated ethylene propylene), PTFE (polytetrafluoroethylene), PVDF (polyvinylidene fluoride), silicone(s), PFA (perfluoroalkoxy alkane(s)) copolymers of tetrafluoroethylene (C₂F₄) and perfluoroethers (e.g., C₂F₃OR^{f}, where R^{f} is a perfluorinated group such as perfluorinated C₁-C₆ alkyl (e.g., trifluoromethyl)), and combinations thereof.

In an embodiment, all or part of the components or surfaces of the ECMO device that contact blood, including tubing, pump components, reservoirs, gas exchange membranes, and/or the housing holding the gas exchange membrane(s) are made from or lined with a fluoropolymer or perfluoropolymer (e.g., FEP, PTFE, and/or PFA). The ECMO apparatus described herein may advantageously be used to treat living subjects (patients) acting to remove carbon dioxide from a subject's blood and to oxygenate it, after which it may be returned to the subject. The apparatus may also be utilized for the perfusion of organs ex-vivo for research and as a means to extend the period the organ(s) remain viable for transplant.

I.1. ECMO Apparatuses Comprising Fluid and Solid Repellant Slippery Surfaces

In an embodiment, the disclosure sets forth methods of treating/preparing an ECMO apparatus such that it comprises a fluid and solid repellant slippery surface on all or part of the surfaces which will contact blood recirculated to a subject. Such methods comprise:
exposing all or part of the apparatus to conditions that produce surface oxygen species capable of reacting with a reactive head group of an anchoring layer molecule (e.g., hydroxyl groups), wherein the anchoring layer molecule comprises the reactive head group and one or more alkyl, fluoroalkyl, or perfluoroalkyl groups, each of which is selected independently;
contacting the anchoring layer molecule and the surface oxygen species, in the presence or absence of a crosslinking agent which is optionally present, to form a covalent bond between the head group of the anchoring layer molecules and the surface oxygen species, thereby forming a surface with covalently bound anchoring layer molecules; removing unreacted anchoring layer molecules; and
contacting at least the part of the apparatus having covalently bound anchoring layer molecules with a lubricating liquid to form a fluid and solid repellant slippery surface.

Fluoroinated and/or perfluorinated lubricating liquids self-assemble on the surfaces treated to form an anchoring layer to form fluid and solid repellant slippery surfaces, particularly where the anchoring layer comprises fluoroalkyl or perfluoroalkyl groups. The lubricating liquid coating remains stably associated with the surface for extended periods due to, for example, hydrophobic interaction, Van der Waals forces, and the relative immiscibility of the lubricating liquid in non-fluorinated medium (e.g., blood). The stability of the lubricating liquid associated with the surface is evidenced by, for example, the continued resistance of the surfaces to bacterial colonization and or biofilm growth even after exposure to flowing blood or saline.

After treatment with the lubricating liquid, any excess lubricating liquid may be drained from the apparatus, allowed to evaporate or carried away by a stream of gas passed over the treated surfaces. The apparatus may also be flushed/rinsed with a liquid (e.g., a bolus of a blood compatible saline, such as phosphate buffered saline or Ringer's lactate) to assist in removal of excess lubricating fluid. In one such embodiment, at least the surface of the membrane that will contact blood during use of the apparatus is treated.

I.2. ECMO Apparatuses Comprising Hydrophobic and/or Omniphobic Surfaces

In an embodiment, the disclosure sets forth methods of treating/preparing an ECMO apparatus such that it comprises a hydrophobic or omniphobic surface on all or part of the surfaces which will contact blood that is recirculated to a subject. Such methods comprise:
optionally exposing all or part of the apparatus to conditions that produce surface oxygen species capable of reacting with a reactive head group of an anchoring layer molecule (e.g., hydroxyl groups), wherein the anchoring layer molecule comprises the reactive head group and one or more alkyl, fluoroalkyl, or perfluoroalkyl groups, each of which is selected independently;
contacting the anchoring layer molecule and the surface oxygen species, in the presence or absence of a crosslinking agent which is optionally present, to form a covalent bond between the head group of the anchoring layer molecules and the surface oxygen species (directly or through the crosslinking molecule), thereby forming a surface with covalently bound anchoring layer molecules; and
removing unreacted anchoring layer molecules.

Exposing all or part of the apparatus to conditions that produce surface oxygen speciescapable of reacting with a reactive head group of an anchoring layer molecule need only be conducted where the surface does not contain functional groups capable of reacting with the reactive head group of an anchoring layer molecule or the cross-linking agent.

When hydrophobic or omniphobic the resulting surface may have a contact angle with water that is greater than 90°, 100°, 110°, 120°, 130°, 140°, or 160° (e.g., from about 90° to about 120°, about 120° to about 140°, about 140° to about 160°, or about 160° to about 175°) measured at 22° C. using a goniometer. When omniphobic the resulting surfaces have a contact angle with dodecane greater than 90°.

I.3. ECMO Apparatuses Comprising a Surface Coated with a Lubricating Liquid

In an embodiment, the disclosure sets forth methods of treating/preparing an ECMO apparatus such that all or part of the surfaces that will contact blood recirculated to a subject are treated with a lubricating liquid. In an embodiment the surfaces of the ECMO apparatus to be treated will be prepared from a fluoropolymer, perfluoropolymer, or a blend of a fluoropolymer and perfluoropolymer. This may be accomplished by making all of the ECMO apparatus or a component of the ECMO apparatus out of a fluoropolymer and/or perfluoropolymer, or coating or lining (e.g., providing a fluoropolymer liner in tubing) all or part of the ECMO apparatus with a fluoropolymer and/or perfluoropolymer. All of the ECMO apparatus, or the portions where it is desirable to have a coating of lubricating liquid (e.g., the portions that contact blood) are then exposed to the lubricating liquid, which can non-covalently bind (become immobilized on the surface) through a combination of forces (e.g., hydrophobic and Van der Waals forces). In an embodiment, a lubricating liquid coating/surface is formed on all or part of an apparatus prepared from a fluoropolymer and/or perfluoropolymer using a fluorinated and/or perfluorinated lubricating liquid. Where the surface is a fluoropolymer and/or a perfluoropolymer and the lubricating liquid is perfluorinated, the surface may be termed a “FILP” (Fluoropolymer and/or perfluoropolymer Immobilized Liquid Perfluorocarbon (or Perfluorinated liquid)) surface. Advantageously, surface treatments, such as exposure to plasma (e.g., oxygen plasma) and application of an anchoring layer, are not required to prepare surfaces coated with a lubricating liquid, particularly FILP coatings.

Fluorinated and/or perfluorinated lubricating liquids self-assemble on fluoropolymer and/or perfluoropolymer surfaces to form lubricating liquid surfaces. The lubricating liquid remains stably associated with the surface for extended periods due to, for example, hydrophobic interaction, Van der Waals forces, and the relative immiscibility of the lubricating liquid in non- fluorinated medium (e.g., blood). The stability of the lubricating liquid associated with the surface is evidenced by, for example, the continued resistance of the surfaces to bacterial colonization and or biofilm growth even after exposure to flowing blood.

In an embodiment, methods to prepare lubricating liquid surfaces (e.g., FILP surfaces) comprise contacting with a lubricating liquid all or part of the ECMO apparatus that contacts a subject's blood to form a lubricating liquid treated surface. After treatment with the lubricating liquid, any excess lubricating liquid may be drained from the apparatus, allowed to evaporate or carried away by a stream of gas passed over the treated surfaces. The apparatus may also be flushed/rinsed with a liquid (e.g., a bolus of a blood compatible saline, such as phosphate buffered saline or Ringer's lactate) to assist in removal of excess lubricating fluid. In one such embodiment, at least the surface of the membrane that will contact blood during use of the apparatus is treated with the lubricating liquid.

Different portions (e.g., components such as tubes, membranes, housings, or pumps) of an ECMO apparatus may be subject to different treatments and bear one or more of a fluid and solid repellant slippery surface, a hydrophobic or omniphobic surface, and/or a lubricating liquid treated surface. As discussed above, the methods described herein and apparatuses resulting from those methods may have the entire surface that will be exposed to a patient or subject's blood (the ECMO circuit) treated. Where less than all of the components of an ECMO apparatus are treated, the treatment may be applied to, for example, (i) the membrane, (ii) the membrane and portions of the ECMO apparatus other than the lines (tubing and/or catheters) that connect the device to the patient or subject), (iii) the lines (tubing and/or catheter), and/or (iv) any pump components that contact blood. Different components of an ECMO apparatus (along with the associated lines) may be treated individually and assembled into a complete unit and, as such, individual components may be treated using different procedures to effectively apply either the same or a different type of surface treatment. By way of example, in an embodiment an ECMO apparatus, other than the associated tubing/catheters, is treated to comprise a fluid and solid repellant slippery surface, while the tubing and catheters are treated to have their internal surface that will contact blood become omniphobic. In another embodiment, an ECMO apparatus is treated such that at least the membrane (e.g., a membrane comprising poly-4-methyl pentene) has a fluid and solid repellant slippery surface, a hydrophobic or omniphobic surface, or a lubricating liquid treated surface.

### II. Preparation of ECMO Apparatus

II.1 Polymers, Fluoropolymers, and Perfluoropolymers for Forming ECMO Apparatus

A diverse group of polymers, fluoropolymers and/or perfluoropolymers may be utilized to prepare the ECMO apparatus or components thereof described herein including the gas exchange membrane(s). The polymers include, for example, polyolefins (e.g., poly-4-methyl pentene, polyethylene, or polypropylene), polyurethanes, polyvinylchloride (PVC), polyvinylidene fluoride, silicone and the like.

Where fluoropolymers and/or perfluoropolymers are employed they may be selected from a wide range of materials including, but not limited to, perfluoroalkoxy alkanes (PFA or PFAs when plural); polytetrafluoroethylene (PTFE); fluorinated ethylene propylene (FEP); expanded polytetrafluoroethylene (ePTFE or EPTFE); expanded fluorinated ethylene propylene (eFEP or EFEP); perfluoromethylvinylether (PMVE); perfluoro elastomers (e.g., FFKM, which are copolymers of tetrafluoroethylene and a perfluorinated ether such as PMVE sold under the tradename TECNOFLON®, TECNOFLON® PFR branded as KALREZ®, CHEMRAZ® and PERLAST®) and combinations thereof. Fluoropolymers that may be employed include, but are not limited to, ethylene tetrafluoroethylene (ETFE); polyvinylidene fluoride (PVDF); fluoroelastomers: (FKM and FEPM sold under the tradenames VITON®, TECNOFLON®); vinylidene fluoride-hexafluoropropylene fluoroelastomer (VF2/HFP); vinylidene fluoride-hexafluoropropylene/tetrafluoro ethylene/hexafluoropropylene fluoroelastomer (VF2/tetrafluoro ethylene/HFP) terpolymer; and combinations thereof.

In embodiment, any component that contacts blood, other than the gas exchange membrane of the ECMO apparatus, may be made of 98%-100% silicone, PTFE, PFA, FEP, PVDF, or a blend of PTFE, PFA, FEP and/or PVDF, where the blend contains two, three or all four types of those fluoropolymers. The individual fluoropolymers in the blend may be present in a range of 2-10, 10-15, 15-20, 20-30, 30-40, 40-50, 50-60, 60-70, 70-80, 80-90, or 90-98 weight % (the composition totaling 100 weight %). In an embodiment blood perfusion tubing connected to the ECMO apparatus is comprised of silicone, polyurethane, PVC, or a PVC blended with PTFE, PFA, FEP, and/or PVDF, where the tubing may be coated or lined with PTFE, PFA, FEP, and/or PVDF (e.g., to facilitate formation of a lubricated liquid surface when the tubing is contacted with fluorinated or perfluorinated lubricating liquid). In an embodiment blood perfusion tubing connected to the ECMO apparatus comprises 98%-100% silicone, PTFE, PFA, FEP, PVDF, or a blend of PTFE, PFA, FEP, and/or PVDF, where the blend contains two, three or all four types of the fluoropolymers. Individual fluoropolymers in the blend may be present in a range of 2-10, 10-15, 15-20, 20-30, 30-40, 40-50, 50-60, 60-70, 70-80, 80-90, or 90-98 weight % (the composition totaling 100 weight %).

Gas exchange membranes for the ECMO apparatus may comprise polymers such as polyolefins (e.g., polypropylenes or polyethylenes) and silicones, alone or in combination with fluoropolymers and/or perfluoropolymers. In an embodiment, ECMO apparatus gas exchange membranes may comprise silicone, a fluoropolymer and/or a perfluoropolymer (e.g., PTFE, PFA, FEP, PVDF) or a blend of any two, three or more fluoropolymers and/or a perfluoropolymers (e.g., PTFE, PFA, FEP and/or PVDF). In an embodiment, the membranes are in the form of one or more hollow fibers.

In an embodiment, ECMO apparatus gas exchange membranes comprise 98%-100% silicone, PTFE, PFA, FEP, PVDF, or a blend of PTFE, PFA, FEP, and/or PVDF, where the blend contains two, three or all four types of the fluoropolymers. Individual fluoropolymers in the blend may be present in a range of 2-10, 10-15, 15-20, 20-30, 30-40, 40-50, 50-60, 60-70, 70-80, 80-90, or 90-98 weight % (the composition totaling 100 weight %). In an embodiment, the oxygenator membrane is in the form of one or more hollow fibers.

In an embodiment, ECMO apparatus gas exchange membrane(s) are made of a polyolefin or a blend of polyolefins including polyethylene, polypropylene or polymethylpentene with either of silicone, FEP, PFA, PTFE, PVDF or a blend of those fluoropolymers. The silicone, or the individual fluoropolymers, may be present in a range of 2-10, 10-15, 15-20, 20-30, 30-40, 40-50, 50-60, 60-70, 70-80, 80-90, 90-98 weight % (the composition totaling 100 weight %). In an embodiment, the ECMO apparatus gas exchange membrane is in the form of one or more hollow fibers.

The surface of the ECMO apparatus, or at least a portion of a surface of the apparatus or any one or more components upon which a fluid and solid repellant slippery surface, a hydrophobic or omniphobic surface or a lubricating liquid surface is formed may have a roughness defined by the ratio of the actual surface area divided by the projected surface area that is in a range selected from 1.00 to 1.50 (e.g., 1.00 to 1.01, 1.01 to 1.05, 1.05 to 1.10, 1.10 to 1.2, or 1.2 to 1.5) or less than 1.5 (e.g., less than about 1.4, less than about 1.3, less than about 1,2, or less than about 1.1) times the projected surface area. The treated surfaces may have Ra and/or Rz values less than 30, 25, 20, 15, 10, 5, 4, 3, 2, or 1 micron (e.g., the Ra and/or Rz values may be in a range selected from the group consisting of 0.1 to 25 microns, 0.1 to 5 microns, 5 to 10 microns, 10 to 20 microns, 20 to 25 microns, or 25 to 30 microns. Surface roughness values are measured using a Mahr Pocket Surf® PS1 roughness analyzer according to the manufacturer's instructions (Mahr Federal Inc., Providence, R.I.) with roughness determined in the absence of any lubricating liquid (e.g., fluorinated and/or perfluorinated hydrocarbon). As noted above, the gas exchange membranes are often microporous; however, the polymeric material used for other components may be porous or non-porous.

II2 Preparation of ECMO Apparatus Comprising a Fluid and Solid Repellant Slippery Surface or a Hydrophobic or Omniphobic Surface

Both hydrophobic or omniphobic surfaces and fluid and solid repellant slippery surfaces require the incorporation of a layer of anchoring molecules to alter the surface properties. The anchoring layer molecule(s) comprises a reactive head group and one or more alkyl, fluoroalkyl, or perfluoroalkyl groups. In order for the reactive head group to covalently attach to polymeric surfaces of the ECMO apparatus in sufficient amounts, reactive species must be incorporated into the polymeric surfaces, after which anchoring layer molecules, alone or in the presence of crosslinkers, may be attached to the polymeric surfaces.

II.2.a Incorporation of Surface Reactive Species

In order to covalently attach anchoring layer molecules to the desired surfaces of the ECMO apparatus, the surfaces must be functionalized with species that can form a covalent bond with the head group of the anchoring layer molecules. Among the potential groups that can be utilized to form a bond with the head groups are amine, sulfhydryl, silanol, and hydroxyl groups. While any number of methods can be used to introduce such groups, or functionalities that give rise to such groups (e.g., peroxides and/or ozonides that can be subsequently converted to hydroxides), exposure to oxygen plasma represents a reproducible means by which to activate all or part of the surface of ECMO devices and their attached tubing and pump components, etc. (e.g., the plastic material). For activation by oxygen plasma, portions of the device (e.g., the membrane) may be exposed to the plasma separately from the remaining components. Alternatively, parts of the device may be masked while other portions are allowed to interact with the plasma. In some instances the ECMO circuit, oxygenator and catheters (tubing, for receiving blood from and returning it to the patient, that is connected directly or indirectly to the inlet or outlet of the ECMO apparatus) are subject to plasma treatment separately.

ECMO apparatus components may be subject to oxygen plasma exposure to determine the exposure to oxygen plasma required to activate the surface for subsequent treatments with anchoring layer molecules. The treatment should be sufficient such that the desired hydrophobicity or ability to form a slippery surface can be achieved. Alternatively, a determination of the exposure to oxygen plasma necessary for activation of the ECMO apparatus surfaces may be conducted using a sheet of plastic (such as PVC or poly-4-methyl pentene) as a calibrator. In an embodiment the sheet of plastic is exposed to 300-400 mTorr of oxygen plasma for a time period sufficient to cause the plastic sheet to have a contact angle with water immediately after plasma exposure selected from the group consisting of about 60° to about 40°, 40° to about 30°, about 30° to about 20°, and 20° to about 10° as measured with a goniometer at 22° C.

Other surface activation methods that provide surface species (e.g., oxygen species) capable of reacting with a reactive head group of an anchoring layer molecule include chemical oxidation using i) peroxide/acid or peroxide/amine mixture, and ii) oxygen plasma cleaning followed by vapor phase treatment with SiCl₄, AlCl₃, or titanium isopropoxide. Where the treatment methods provide active species other than oxygen species that can react with the head group of an anchoring layer molecule, such as for example amine or sulfhydryl groups, the treatment methods and techniques described herein can also be employed.

II.2.b Anchoring Layer Molecules and Their Attachment to Surfaces

An anchoring layer molecule comprises the reactive head group and one or more alkyl, fluoroalkyl, or perfluoroalkyl groups attached to the reactive head group, each of which is selected independently.

Reactive head groups include among other things carboxylic acids (or their acid chlorides or anhydrides), epoxides, aziridines, thiiranes, silanes, isothiocyanates and isocyanates. In certain embodiments the head group of the anchoring layer molecule is selected from: monochloro silanes, dichloro silanes, trichloro silanes, monomethoxy silanes, dimethoxy silanes, trimethoxy silanes, monoethoxy silanes, diethoxy silanes triethoxy silanes, monoformyloxy silanes, diformyloxy silanes, triformyloxy silanes, monoacetoxy silanes, diacetoxy silanes, triacetoxy silanes, and combinations thereof.

In some embodiments the anchoring layer molecules are of the formula RₙSiX₄₋ₙ (Formula I), wherein
n=1, 2 or 3;
X is H, Cl, OCH₃, OCH₂CH₃, OC(O)H, OC(O)CH₃ or a combination thereof; and
R is an alkyl, fluoroalkyl, or perfluoroalkyl group each of which is selected independently.

In such an embodiment R may be limited to fluoroalkyl or perfluoroalkyl, without the presence of alkyl groups. Alternatively, R may be limited to alkyl without the presence of any fluoroalkyl or perfluoroalkyl groups.

In embodiments, including embodiments where the anchoring group molecules are of formula I, at least one alkyl, fluoroalkyl or perfluoroalkyl group present comprises from about 6 to about 16 carbon atoms. For example, the R alkyl, fluoroalkyl or perfluoroalkyl group may comprise from about 6 to about 12 carbon atoms, from about 6 to about 8 carbon atoms, from about 6 to about 9 carbon atoms, from about 8 to about 16 carbon atoms, about 8 carbon atoms, about 7 carbon atoms, or about 6 carbon atoms. Alkyl, fluoroalkyl or perfluoroalkyl groups may also comprise less than 6 or more than 16 carbon atoms.

In another embodiment the anchoring layer molecules are molecules of formula I that comprise at least one, at least two, or three fluorotelomers. In one such embodiment the fluorotelomer(s) are of the formula (CH₂)×(CF₂)_{y}F, where x is 1-5 and y is 1-9. A variety of fluorotelomers that are each selected independently may appear in the molecules of Formula I. Included in the molecules of Formula I are fluorotelomer groups where the combination of x and y is selected from the group consisting of: x is 2 and y is 4; x is 2 and y is 5; x is 2 and y is 6; x is 2 and y is 7; x is 2 and y is 8; x is 4 and y is 4; x is 4 and y is 5; x is 4 and y is 6; x is 4 and y is 7; and x is 4 and y is 8.

II.2.c Crosslinkers

A variety of crosslinking agents may be employed to assist in forming an anchoring layer. The crosslinking agents may serve several roles including, but not limited to, reacting with the anchoring layer molecules to form oligomers that can be attached to the surface of the ECMO apparatus and, where polyvalent, reacting with the active species on the surface of the ECMO apparatus (e.g., active oxygen species from plasma treatment) to provide more sites where anchoring molecules can bind.

In an embodiment the crosslinking agents are of the form (R1O)₃Si—(CH₂)ᵥ—Si(OR1)₃, where v is 1-8 and R1 is alkyl (e.g., methyl or ethyl).

In an embodiment the crosslinking agents are of the form (R2)_{4-w}C[(CH₂)ᵥOH]_{w}, where R2 is H, alkyl, OH, or —(CH₂)ₓOH, w is 2, 3, or 4, x is 1-4, and v is 1-8.

In an embodiment the crosslinking agents are of the form formula (R4O)₃—Si—R3-Si(OR4), where R3 is —(CF₂)₁₋₆— or —(CH₂)₁₋₆—, and R4 is alkyl or fluoroalkyl.

In an embodiment the crosslinking agents are of the form Cl₃Si—R3-SiCl₃, where R3 is —(CF₂)₁₋₆— or —(CH₂)₁₋₆—.

In an embodiment the crosslinking agents are of the form R5(CH₂CH₂)_{y}Si(OR4)₃, where R4 is alkyl or fluoroalkyl (e.g., methyl or ethyl), R5 is —NCO, —NH₂, —NHR4, —N(R4)₂, or epoxy, and y is 1,2,3, or 4.

In an embodiment the crosslinking agent is selected from the group consisting of tetrachloro silane, tetramethoxy silane, and tetraethoxy silane.

II.2.d Contacting Anchoring Layer Molecules with Reactive Species (e.g., Reactive Oxygen Species)

The process of forming a covalent bond between a reactive species on the surface of an ECMO apparatus component and an anchoring layer molecule requires contacting the reactive species and the molecule. This may be done in the presence of crosslinking agents, which are optional. Contacting may be conducted with the anchoring molecule and/or crosslinker in the vapor phase or in a liquid phase.

Processes conducted in the vapor phase may be conducted under reduced pressure or in the presence of an inert and/or dry atmosphere if necessary. For example, (tridecafluoro-1,1,2,2-tetrahydrooctyl) triethoxy silane or (tridecafluoro-1,1,2,2-tetrahydrooctyl) trichlorosilane may be reacted with the moist surface of an ECMO component or the entire ECMO circuit by contacting or passing a gas, vapor, or aerosol comprising those compounds through or over the surfaces to be modified. In an embodiment, a gas, vapor, or aerosol comprising an anchoring molecule (and optionally one or more crosslinking agents) is passed through an entire ECMO circuit thereby treating all of the surfaces that a subject's blood will come into contact with. In another embodiment, the ECMO apparatus or components of the apparatus, are exposed to a gas, vapor, or aerosol comprising (tridecafluoro-1,1,2,2-tetrahydrooctyl) trichlorosilane without being moistened.

The process of forming a covalent bond between a reactive species on the surface of an ECMO apparatus component and an anchoring layer molecule may also be carried out in the liquid phase using aprotic, protic, or a mixture of protic and aprotic liquids.

When using a liquid mixture to contact the anchoring layer molecules with the surface active species (e.g., surface oxygen species) the process may comprise:
(i) contacting a mixture comprising the anchoring layer molecule and an aprotic solvent with all or part of the apparatus, or
(ii) contacting a mixture comprising the anchoring layer molecule and a protic solvent with all or part of the apparatus;

wherein the anchoring layer molecule comprises 1%-99%, 1%-10%, 1%-20%, 1%-50%, 10%-20%, 10-50%, 20%-30%, 30%-40%, 40%-50%, 50%-60%, 60%-70%, 70%-80%, 80%-90%, or 90%-99% of the mixture by weight. While the time to achieve covalent modification of the apparatus may vary, in an embodiment the mixture is contacted with all or part of the apparatus for 1-25 hours (e.g., 1-2, 1-5, 1-10, 1-20, 2-5, 2-10, 2-20, 2-25, 5-10, 5-20, 5-25, 10-20 or 10-25 hours). In an embodiment, the mixture with the anchoring layer molecule, whether comprising a protic or aprotic solvent, comprises from about 1% to about 20%, 1% to 5%, 5% to 10%, or 10% to 20% of the anchoring layer molecules by weight; and optionally comprises 0 to about 5%, 0.1% to 0.2%, 0.2% to 0.5%, 0.5% to 2.0%, or 2.0% to 5.0%, of a crosslinker by weight.

In an embodiment the mixture contacted with all or part of the ECMO apparatus comprises the anchoring layer molecule and the aprotic solvent, with the aprotic solvent comprising a solvent selected from the group consisting of: dioxanes, toluene, benzene, para-chlorobenzenetrifluoride, tetrahydrofuran (THF), CHCl₃, CH₂Cl₂, perfluorinated alkanes (e.g., perfluorodecalin), tri(fluoroalkyl)amines, and mixtures thereof.

In an embodiment the mixture contacted with all or part of the ECMO apparatus comprises the anchoring layer molecule and the protic solvent, with the protic solvent comprising a solvent selected from the group consisting of: methanol, ethanol, isopropanol, butanol, perfluorobutanol, fluorinated alcohols of the formula CF₃(CF₂)ₚ(CH₂CH₂)_{q}—OH where p=1, 2, 3, 4, 5, 6, or 7 and q=0, 1, or 2), and mixtures thereof. In such an embodiment the protic solvent may further comprise water and/or an aprotic solvent. Water or aprotic solvents, when present, may in some embodiments comprise less than 80%, 70%, 60%, 50%, 40%, 30%, 20%, 10%, 5%, or 1%. Some aprotic solvents that may be used with the protic solvent and/or water include dioxanes, toluene, benzene, para-chlorobenzenetrifluoride, tetrahydrofuran (THF), CHCl₃, CH₂Cl₂, perfluorinated alkanes (e.g., perfluorodecalin), tri(fluoroalkyl)amines, and mixtures thereof.

Following treatment (contacting) with a mixture comprising an anchoring layer molecule in the liquid phase, the mixture is drained from the apparatus. In an embodiment, the apparatus is dried to a substantially constant weight under a stream of gas. Suitable gases include, but are not limited to, air, nitrogen, oxygen, carbon dioxide, an inert gas (e.g., helium, neon, argon), or a mixture thereof.

In an embodiment, following contacting the anchoring layer molecules (and crosslinker if present) with the surface oxygen species to form a covalent bond between the head group of the anchoring layer molecules and the surface, the apparatus is cured at a temperature greater than about 40° C. Some suitable temperatures for curing include, but are not limited to, about 40° C. to about 72° C., about 40° C. to about 60° C., or about 50° C. to about 70° C. Depending on the combination of the anchoring layer molecule and the reactive group the curing time may vary from about 4 hours to about 48 hours, about 4 hours to about 10 hours, about 10 hours to about 20 hours, about 10 hours to about 48 hours, about 20 hours to about 30 hours, about 20 hours to about 48 hours, about 30 hours to about 40 hours, or about 30 hours to about 48 hours.

Crosslinking agents may also be present when contacting the anchoring layer molecule and the surface reactive species (e.g., oxygen species). In embodiments, the crosslinking agent(s) are selected independently from the group consisting of:
i) one or more independently selected crosslinking agents of the formula (R1O)₃Si—(CH₂)ᵥ—Si(OR1)₃, where v is 0-8, and R1 is alkyl;
ii) one or more independently selected crosslinking agents of the formula (R2)_{4-w}C[(CH₂)ᵥOH]_{w}, where R2 is H, alkyl, OH, or —(CH₂)ₓOH, where w is 2, 3, or 4, x is 1-4, and v is 0-8;
iii) one or more independently selected crosslinking agents of the formula (R4O)₃—Si—R3-Si(OR4), where R3 is —(CF₂)₁₋₆— or —(CH₂)₁₋₆—, and R4 is alkyl or fluoroalkyl;
iv) one or more independently selected crosslinking agents of the formula Cl₃Si—R3-SiCl₃, where R3 is —(CF₂)₁₋₆— or —(CH₂)₁₋₆—; and
v) one or more independently selected crosslinking agents of the formula R5(CH₂CH₂)_{y}Si(OR4)₃, where R4 is alkyl or fluoroalkyl, R5 is —NCO, —NH₂, —NHR4, —N(R4)₂, or epoxy, and y is 1, 2, 3, or 4.

In one such embodiment, the crosslinking agent comprises at least one crosslinker from group (i) and group (ii).

### III. Lubricating Liquids

III.1 Types of Lubricating Liquids

Both fluid and solid repellant slippery surfaces and lubricating liquid surfaces present a lubricating liquid as the interface with blood. Their preparation requires bringing a lubricating liquid (e.g., a fluoro and/or perfluorohydrocarbon) into contact with a surface comprising an anchoring layer when preparing a fluid and solid repellant slippery surface, or a polymer (e.g., a fluoropolymer and/or perfluoropolymer component or layer on a component) when making a lubricating liquid surface. Virtually any suitable liquid that will stably associate with the anchoring layer (in the case of a fluid and solid repellant slippery surface) or the polymer to which it is applied (in the case of a lubricating liquid surface) through noncovalent interactions such as those resulting from van der Waals forces and hydrophobic interactions may be employed.

In an embodiment, the lubricating liquid comprises one or more independently selected alkanes, fluorinated hydrocarbons, perfluorinated hydrocarbons, fluorinated alkanes, fluorinated alkylethers, perfluorinated alkanes, tri(perfluoroalkyl)amines, perfluoropolyethers or a combination thereof. Such combinations include, for example:
i) one or more alkanes, fluorinated alkanes, perfluorinated alkanes, tri(perfluoroalkyl)amines, and combinations thereof;
ii) one or more fluorinated alkanes, perfluorinated alkanes, tri(perfluoroalkyl)amines, and/or combinations thereof;
iii) one or more fluorinated alkanes, perfluorinated alkanes, and combinations thereof;
iv) one or more perfluorinated alkanes, tri(perfluoroalkyl)amines, and/or combinations thereof; and
v) one or more alkanes, fluorinated alkanes, perfluorinated alkanes, and/or combinations thereof.

In an embodiment, the lubricating liquid comprises a liquid selected from the group consisting of perfluorotripropylamine, perfluorotripentylamine, perfluorotributylamine, perfluorodecalin, perfluoromethyldecalin, perfluorooctane, perfluorobutane, perfluoropropane, perfluoropentane, perfluorohexane, perfluoroheptane, perfluorononane, perfluorodecane, perfluorododecane, perfluorooctyl bromide, perfluoro(2-butyl-tetrahydrofurane), perfluoroperhydrophenanthrene, perfluoroethylcyclohexane, perfluoro(butyltetrahydrofuran), perfluoropolyethers (KRYTOX™), 3-ethoxy-1,1,1,2,3,4,4,5,5,6,6,6-dodecafluoro-2-trifluoromethylhexane, trifluoromethane, difluoromethane, pentafluoroethane, or a combination thereof.

In an embodiment the lubricating liquid comprises butane, pentane, hexane, cyclohexane, heptane, octane, nonane, decane, dodecane, hexadecane, octadecane, triacylglycerides, polydimethylsiloxane, polydiethylsiloxane, fluorinated siloxanes (e.g., (CH₃)₃Si—O—(Si[CH₂CH₂(CF₂)₀₋₅CF₃]₂—O—Si(CH₃)₂)₁₋₂₀—O—Si(CH₃)₃), mineral oil, alkenes, cholesterol, or a combination thereof. Each of these components may be used in combination with any of the other lubricating liquids.

III.2 Application of Lubricating Liquids to ECMO Apparatus

Lubricating liquids may be brought into contact with a surface upon which a fluid and solid repellant slippery surface, or a lubricating liquid surface, is to be formed using any method known in the art including, but not limited to, spraying, exposure to a vapor or mist containing the lubricating liquid, painting, dipping, filling, and the like. The ECMO apparatus may be contacted with the lubricating liquid as an assembled unit, or separate components that will make up the ECMO apparatus may be separately treated and assembled into a complete ECMO apparatus. For example, the housing containing the gas exchange membrane may be prepared and contacted with lubricating liquids separately from other components that may be incorporated into an ECMO apparatus (e.g., the housing and membrane may be subject to oxygen plasma, reacted with anchoring molecules, and contacted with a lubricating liquid separately from tubing, revivor(s), pump(s) etc.).

In an embodiment, the lubricating liquid is introduced into the ECMO apparatus such that it fills or passes as a bolus through the portion of the apparatus that blood will occupy when the apparatus is in use (e.g., it fills or contacts the first portion of the chamber including the inner walls and membrane(s), as well as any associated channels through which blood flows). The excess fluid is then drained from the apparatus and/or forced from the apparatus using a stream of gas and/or fluid such as a sterile gas, water, or saline. Passage of a stream of gas can assist in evaporating excess fluid in preparation for storing the apparatus “dry.” Passage of fluids can remove excess lubricating liquid and prepare the apparatus for storage in a fluid primed state. Fluid used to remove excess lubricating liquid from an ECMO apparatus (or a component of an ECMO apparatus) can be drained from the apparatus prior to introducing blood into the apparatus, or can be displaced by a desired priming solution (e.g., Ringer's lactate) or blood.

### IV. Properties of the ECMO Apparatus and Their Surfaces

The lubricating liquid surfaces, hydrophobic or omniphobic surfaces, and fluid and solid repellant slippery surfaces of an ECMO apparatus treated as described herein do not stimulate clotting to the same degree as do the untreated surfaces. This reduction in blood coagulation is observed whether the treated surfaces are fluid and solid repellant slippery surfaces, hydrophobic or omniphobic surfaces, or lubricating liquid treated surfaces. Accordingly, ECMO apparatuses may be operated using less anticoagulants than would be possible using an otherwise identical ECMO apparatus lacking the surface treatment.

The thrombogenicity or attachment of clotted blood, and/or thrombi formation associated with the treated surfaces, may be determined by any suitable method known in the art. In an embodiment, the amount of clotted blood attached to a surface may be determined by measurement of a blood specific component, such as a protein or portion of a protein, that has attached to the surface. In an embodiment, the protein may be hemoglobin. In another embodiment the protein may be fibrin (as opposed to its soluble form fibrinogen). In another embodiment, the heme (porphyrin) group of hemoglobin may be measured (e.g., following proteolytic digestion of the adherent clot with trypsin). In an embodiment, the fraction of the surface area upon which thrombus formation occurs may be used as a measure of thrombogenicity.

Where a thrombogenicity formation score based on the area subject to thrombus formation is used as a measure of thrombogenicity, a sample of material may be formed as a tube with the test surface on its inner wall and tested as part of a closed test loop of tubing through which blood is circulated. Where the material is not amenable to preparation in the form of a tube, a section of the material may be inserted in the test loop and blood circulated in the test loop. For such tests a sample of blood is drawn from a mammal (a sheep, unless stated otherwise) and is treated with 1 Howell unit of heparin per ml. Subsequent studies utilizing the heparinized blood are initiated within 30 minutes of the blood draw. The Activated Clotting Time (ACT) is between 150 and 250 seconds.

A 140 cm overall length test loop (about 9.5 mm inside diameter) is prepared from a length of additional tubing (e.g., LivaNova® perfusion pack tubing) to which a tube of the test material is attached or into which a test sample about 2.5 cm long is inserted. The test loop is filled with blood (sheep blood, unless stated otherwise) heparinized as described above. Samples are run in groups of three replicates. Blood is circulated through the loop using a peristaltic pump on a portion of the tubing that is not formed from the test material at 37±1° C. for 4 hours±30 minutes.

At the end of the 4-hour period the sample is washed gently with normal saline so as not to remove blood that has clotted and attached to the sample and the samples are photographed. If the sample is formed as a tube that is not transparent, it may be sliced open for visualization of the inner surface which was exposed to blood. Samples are scored on a scale of 1-5 as indicated in Table 1.

TABLE 1

| Thrombus | | |
|---|---|---|
| Formation Score | Score Criterion | Thrombogenicity Evaluation |
| 0 | Minimal to nonexistent thrombus formation | Thromboresistant |
| | (1% or less) | |
| 1 | Minimal - Thrombus observed to be covering | |
| | 2 to 10% of material length/area | |
| 2 | Mild - Thrombus observed to be covering 11 | |
| | to 25% of material length/area | |
| 3 | Moderate - Thrombus observed to be | Suspected Thrombogenic |
| | covering 26 to 50% of material length/area | (further evaluation recommended) |
| 4 | Extensive - Thrombus observed to be | Thrombogenic |
| | covering 51 to 75% of material length/area | |
| 5 | Severe - Thrombus observed to be covering | |
| | >75% of material length/area | |

Note:
This test is determining material mediated adherent thrombus formation, therefore only thrombus observed that is adhered to the article is used to determine the score.

Suitable lubricating liquid surfaces, hydrophobic or omniphobic surfaces, and fluid and solid repellant slippery surfaces have a score of 3 or less (e.g., 3, 2, 1, or 0). In an embodiment, lubricating liquid surfaces, and particularly FILP surfaces, have a score of 2 or less or 1 or less (e.g., 2, 1, or 0). In an embodiment, lubricating liquid surfaces, and particularly FILP surfaces, have a score of 2 or less, or 1 or less (e.g., 2, 1, or 0). In an embodiment, fluid and solid repellant slippery surfaces have a score of 2 or less, or 1 or less (e.g., 2, 1, or 0).

In addition to their reduced coagulation induction, the treated surfaces of the ECMO apparatus may have a variety of properties including hydrophobicity or hydrophobicity and oleophobicity (omniphobicity). Oleophobicity and omniphobicity are achieved through the use of fluorinated/perfluorinated molecules as the anchoring layer molecules anchor lubricating liquids.

Once formed, the treated surfaces, and particularly the fluid and solid repellant slippery surfaces, and lubricating liquid surfaces (e.g., FILP fluoropolymer and/or perfluoropolymer surfaces treated with fluorinated and/or perfluorinated liquids) resist the colonization and formation, growth, or attachment of biofilms, formation or attachment of blood proteins, clotted blood, and/or thrombi formation induced when exposing the treated surface to blood (e.g., mammalian blood such as human, porcine, ovine, bovine, canine, feline, equine, etc.). The ability to resist colonization and biofilm formation by bacteria and fungi is important to preventing blood stream infections (e.g., Central Line Associated Blood Stream Infection (CLABSI). The ability of surfaces to resist colonization and/or bioformation after being subjected to conditions that may remove or otherwise degrade surface treatments can be used as a measure of the surface treatment durability. Where the surface is a fluid and solid repellant slippery surface or a lubricating liquid surface (e.g., FILP fluoropolymer and/or perfluoropolymer surfaces treated with fluorinated and/or perfluorinated liquids), the resistance to colonization and/or biofilm growth may be used as a measure of the ability of the lubricating liquid's ability to remain associated with the surface when compared to an otherwise identical surface that has not been treated with the lubricating liquid.

Colonization and/or biofilm formation by bacteria and fungi, and the stable association of lubricating liquids with fluid and solid repellant slippery surface or a lubricating liquid surface (e.g., FILP surfaces) may be assessed in a Flow Loop Durability Study by exposing a sample surface to flowing sterile buffered saline for an initial test period and then subjecting the test surface to a challenge in a growing bacterial culture.

For testing six pre-sterilized (ethylene oxide “ETO”, 12 hours) sections of silastic tubing (LivaNova, Arvada, Colo., 6.3 mm inner diameter and 170 cm length) are filled with sterile phosphate buffered saline (PBS. Sigma. St. Louis Mo.). Test material is sterilized by exposure to ETO for 12 hours in sterilization packets and the ETO sterilized packets opened in a biosafety level (“BSL”) II cabinet, after which they are contacted with 0.2 micron filter sterilized lubricating liquid (e.g. fluorinated or perfluorinated lubricant) as needed for the test sample. The section(s) of sterile test material are then inserted into the PBS filled sterile tubing and all six sections of tubing are closed to form six individual loops avoiding the introduction of any air bubble bigger than ˜3 mm diameter. Three replicates of pre-sterilized test material (e.g., tubing with a FILP coating on its inner surface) and three replicates of otherwise identical control materials that are not subject to surface treatment are thus prepared for the initial phase of testing. This entire loop assembly is performed in a biosafety level (“BSL”) II cabinet. PBS buffer is flowed through all loops using a multichannel peristaltic pump at 50 mL/min flow rate while the loops are submerged in a water bath constantly maintaining 37° C. At every desired time point (e.g., 10, 20, 30, 40, 50, 60, 70, 80, or 90 days), one loop is disengaged and sample(s) are removed from the loop while they are submerged in sterile PBS buffer. Samples are immediately transferred into 5 ml of liquid nutrient broth (Carolina Biological Supply Company, Item #776380) supplemented with 0.5 ml of sterile D-glucose (2.5 M) containing 10⁵ Staphylococcus epidermidis (ATCC® 14990™, American Type Culture Collection (ATCC), Manassas, Va.) colony forming units (CFU). The bacteria are allowed to grow for 48 hours at 37° C., in capped 50 ml culture tubes during which time the media becomes cloudy. At 48 hours samples are removed from the culture media, rinsed by dipping in 2.0 ml of sterile nutrient broth, and placed in tubes containing 1.5 ml of sterile nutrient broth. Samples are vortexed for 5 minutes and subjected to sonication for 1 minute to dislodge any S. epidermidis and disperse it in the nutrient broth. The number of viable S. epidermidis cells released from the samples is determined by serial dilution and plating on 50 mm tryptic soy broth agar plates and/or by serial dilution and measurement of ATP concentration using a BACTITERGLO™ assay from Promega Corp (Madison, Wis.).

Fluid and solid repellant slippery surfaces and lubricating liquid surfaces (e.g., FILP surfaces) show greater than 80% reduction (e.g., about an 85% or a 90% reduction) in bacteria associated with the test samples relative to an otherwise identical sample that is not treated with the lubricating liquid even after 10, 20, 30, 40, 50 or 60 days of exposure to flowing PBS. The test results indicate not only the resistance to bacterial colonization/biofilm growth, but also the stable association of the lubricating liquids with the treated surfaces. The resistance is independent of the roughness of the perfluoropolymer and/or fluoropolymer surface (measured in the absence of any liquid) over the range of surface roughness ratio (actual surface area divided by the projected surface) from 1.0 to 1.5 (e.g., 1.01-1.5), particularly where the surface is covered by fluorinated and/or perfluorinated liquids.

In an embodiment, ECMO apparatus surfaces prepared with fluid and solid repellant slippery surfaces or a lubricating liquid surface (e.g., a FILP surface) resist the growth and attachment of bacteria. The surfaces may be resistant to the growth and attachment of bacteria including, but not limited to, Actinobacillus, Acinetobacter (e.g., Acinetobacter baumannii), Aeromonas, Bordetella, Brevibacillus, Brucella, Bacteroides, Burkholderia, Borrelia, Bacillus, Campylobacter, Capnocytophaga, Cardiobacterium, Citrobacter, Clostridium, Chlamydia, Eikenella, Enterobacter, Escherichia, Francisella, Fusobacterium, Flavobacterium, Haemophilus, Helicobacter, Kingella, Klebsiella, Legionella, Listeria, Leptospirae, Moraxella, Morganella, Mycoplasma, Mycobacterium, Neisseria, Pasteurella, Proteus, Prevotella, Plesiomonas, Pseudomonas, Providencia, Rickettsia, Stenotrophomonas, Staphylococcus (e.g., Staphylococcus epidermidis), Streptococcus (group A), Streptococcus agalactiae (group B), Streptococcus bovis, Streptococcus pneumoniae, Streptomyces, Salmonella, Serratia, Shigella, Spirillum, Treponema, Veillonella, Vibrio, Yersinia, Xanthomonas, and combinations thereof.

In an embodiment, ECMO apparatus surfaces prepared with a fluid and solid repellant slippery surface or a lubricating liquid surface (e.g., a FILP surface) resist the formation, growth, and attachment of fungi including, but not limited to, Aspergillus, Blastomyces dermatitidis, Candida, Coccidioides immitis, Cryptococcus, Histoplasma capsulatum var. capsulatum, Histoplasma capsulatum var. duboisii, Paracoccidioides brasiliensis, Sporothrix schenckii, Absidia corymbifera; Rhizomucor pusillus, Rhizopus arrhizous, and combinations thereof.

In an embodiment, ECMO apparatus surfaces prepared with a fluid and solid repellant slippery surface or a lubricating liquid surface (e.g., a FILP surface) resist the formation, growth, and attachment of viruses including, but not limited to, cytomegalovirus (CMV), dengue, Epstein-Barr, Hantavirus, human T-cell lymphotropic virus (HTLV I/II), Parvovirus, hepatitis, human papillomavirus (HPV), human immunodeficiency virus (HIV), acquired immunodeficiency syndrome (AIDS), respiratory syncytial virus (RSV), Varicella zoster, West Nile, herpes, polio, smallpox, yellow fever, rhinovirus, coronavirus, Orthomyxoviridae (influenza viruses), and combinations thereof.

### EMBODIMENTS

1. A method of preparing all or part of an extracorporeal membrane oxygenator (“ECMO”) apparatus (e.g., its oxygenator) comprising a lubricating liquid surface, the method comprising:
contacting all or part of the apparatus with a lubricating liquid to form a lubricating liquid surface on all or part (e.g., one or more parts that contact blood) of the ECMO apparatus.

2. A method of preparing all or part of an ECMO apparatus (e.g., its oxygenator) comprising a hydrophobic or omniphobic surface, the method comprising:
exposing all or part (e.g., one or more parts that contact blood) of the apparatus to conditions that produce surface oxygen species capable of reacting with a reactive head group of an anchoring layer molecule (e.g., hydroxyl groups), wherein the anchoring layer molecule comprises the reactive head group and one or more alkyl, fluoroalkyl, or perfluoroalkyl groups, each of which is selected independently;
contacting the anchoring layer molecule and the surface oxygen species, in the presence or absence of a crosslinking agent which is optionally present, to form a covalent bond between the head group of the anchoring layer molecules and the surface oxygen species (directly or through the crosslinking molecule), thereby forming a surface with covalently bound anchoring layer molecules; and
removing unreacted anchoring layer molecules.

3. A method of preparing all or part of an ECMO apparatus (e.g., its oxygenator) with a fluid and solid repellant slippery surface, the method comprising:
exposing all or part (e.g., one or more parts that contact blood) of the apparatus to conditions that produce surface oxygen species capable of reacting with a reactive head group of an anchoring layer molecule (e.g., hydroxyl groups), wherein the anchoring layer molecule comprises the reactive head group and one or more alkyl, fluoroalkyl, or perfluoroalkyl groups, each of which is selected independently;
contacting the anchoring layer molecule and the surface oxygen species, in the presence or absence of a crosslinking agent which is optionally present, to form a covalent bond between the head group of the anchoring layer molecules and the surface oxygen species (directly or through the crosslinking molecule), thereby forming a surface with covalently bound anchoring layer molecules;
removing unreacted anchoring layer molecules; and
contacting at least the part of the apparatus having covalently bound anchoring layer molecules with a lubricating liquid to form a fluid and solid repellant slippery surface.

4. A method of preparing all or part of an ECMO apparatus (e.g., its oxygenator) having portions thereof with two or more of (i) a surface coated with a lubricating liquid, (ii) a fluid and solid repellant slippery surface, and (iii) a hydrophobic or omniphobic surface, the method comprising:
treating a portion of the ECMO apparatus to form the lubricating liquid surface as in embodiment 1, treating a portion of the ECMO apparatus to form a hydrophobic or omniphobic surface as in embodiment 2, and/or treating a portion of the ECMO apparatus to form a fluid and solid repellant slippery surface as in embodiment 3.

5. The method of any preceding embodiment, wherein the ECMO apparatus comprises one or more of a polymer, fluoropolymer and/or perfluoropolymer.

6. The method of any preceding embodiment, wherein the membrane comprises one or more of a polymer (e.g., a polymeric material such as silicone or polyolefin (e.g., polyethylene or polypropylene)), fluoropolymer and/or perfluoropolymer.

7. The method of any preceding embodiment, wherein the membrane comprises one or more of poly-4-methyl pentene, propylene, FEP, PTFE, PVDF, silicone, and/or PFA.

8. The method of embodiment 6, wherein the membrane comprises one or more fluoropolymers or perfluoropolymers.

9. The method of embodiment 6, wherein the membrane comprises one or more of FEP, PTFE, PVDF, silicone, and/or PFA.

10. The method according to any of embodiments 5 to 9, wherein all or a portion of the ECMO apparatus comprises a lubricating liquid surface, wherein any portion of the apparatus containing the lubricating liquid has not been subject to activation by plasma etching (e.g., oxygen plasma exposure) and does not contain an anchoring layer applied to the polymer, fluoropolymer and/or perfluoropolymer.

11. The method of embodiment 10, wherein at least the membrane comprises a lubricating liquid surface.

12. The method of embodiment 10 or embodiment 11, wherein the lubricating liquid comprises a fluorinated and/or a perfluorinated liquid (e.g., a fluorocarbon, perfluorocarbon, tri(fluoroalkyl)amine, or a combination thereof).

13. The method of any of embodiments 2 to 9, wherein exposing all or part of the apparatus to conditions that produce surface oxygen species comprises exposing all or part of the apparatus to oxygen plasma.

14. The method of embodiment 13, wherein the oxygen plasma is contacted with all or part of the apparatus at 300-400 mTorr of pressure.

15. The method of embodiment 13 or embodiment 14, wherein all or part of the apparatus is exposed to oxygen plasma in an amount sufficient to produce a sheet of polyvinylchloride (PVC), with a water contact angle in a range selected from the group consisting of 60°-40°, 40°-30°, 30°-20°, and 20°-10° measured at 22° C., immediately following oxygen plasma exposure.

16. The method of embodiment 13 or embodiment 14, wherein all or part of the apparatus is exposed to oxygen plasma in an amount sufficient to produce a sheet of poly-4-methyl pentene, with a water contact angle in a range selected from the group consisting of 70°-60°, 60°-50°, 50°-40°, 40°-30°, 30°-20°, 20°-10° or 10°-0° measured at 22° C., immediately following oxygen plasma exposure.

17. The method of any of embodiments 13 to 16, wherein the head group of the anchoring layer molecule is selected from: hydrogen, monochloro silanes, dichloro silanes, trichloro silanes, monomethoxy silanes, dimethoxy silanes, trimethoxy silanes, monoethoxy silanes, diethoxy silanes, triethoxy silanes, monoformyloxy silanes, diformyloxy silanes, triformyloxy silanes, monoacetoxy silanes, diacetoxy silanes, triacetoxy silanes, and combinations thereof.

18. The method of any of embodiments 13 to 17, wherein the anchoring layer molecules are of the formula RₙSiX₄₋ₙ, wherein
n=1, 2 or 3;
X is H, Cl, —OCH₃, —OCH₂CH₃, —OC(O)H, —OC(O)CH₃, —N(CH₃)₂, —O—N═C(CH₃)₂ or a combination thereof; and
R is an alkyl, fluoroalkyl, or perfluoroalkyl group, each of which is selected independently.

19. The method of any of embodiments 13 to 18, wherein each of the alkyl, fluoroalkyl or perfluoroalkyl groups comprises from about 6 to about 16 carbon atoms, from about 6 to about 12 carbon atoms, from about 6 to about 9 carbon atoms, from about 6 to about 8 carbon atoms, about 8 carbon atoms, about 7 carbon atoms or about 6 carbon atoms.

20. The method of any of embodiments 13 to 19, wherein the anchoring layer molecules comprise fluoroalkyl groups and/or perfluoroalkyl groups.

21. The method of embodiment 20, wherein the fluoroalkyl groups comprise one or more fluorotelomers.

22. The method of embodiment 21, wherein the fluorotelomers are of the formula —(CH₂)ₓ(CF₂)_{y}F where x is 1-5 and y is 1-9.

23. The method of embodiment 22, wherein the combination of x and y is selected from the group consisting of: x is 2 and y is 4; x is 2 and y is 5; x is 2 and y is 6; x is 2 and y is 7; x is 2 and y is 8; x is 4 and y is 4; x is 4 and y is 5; x is 4 and y is 6; x is 4 and y is 7; and x is 4 and y is 8.

24. The method of any of embodiments 13 to 23, wherein contacting the anchoring layer molecules and optional crosslinking agent with the surface oxygen species comprises exposing all or part of the apparatus to the anchoring layer molecules or anchoring layer molecules and crosslinking agent in the vapor phase.

25. The method of any of embodiments 13 to 23, wherein contacting the anchoring layer molecules with the surface oxygen species comprises:
(i) contacting a mixture comprising the anchoring layer molecules and an aprotic solvent with all or part of the apparatus, or
(ii) contacting a mixture comprising the anchoring layer molecules and a protic solvent with all or part of the apparatus;

wherein the anchoring layer molecules comprise 1%-10%, 1%-20%, 1%-50%, 10%-20%, 10-50%, 20%-30%, 30%-40%, 40%-50%, 50%-60%, 60%-70%, 70%-80%, 80%-90%, or 90%-99% of the mixture by weight.

26. The method of embodiment 25, wherein the mixture comprises the anchoring layer molecules and the aprotic solvent, wherein the aprotic solvent comprises a solvent selected from the group consisting of: dioxanes, toluene, benzene, para-chlorobenzenetrifluoride, THF, CHCl₃, CH₂Cl₂, perfluorinated alkanes (e.g., perfluorodecalin), tri(fluoroalkyl)amines, and mixtures thereof.

27. The method of embodiment 25, wherein the mixture comprises the anchoring layer molecules and the protic solvent, wherein the protic solvent comprises a solvent selected from the group consisting of: methanol, ethanol, isopropanol, butanol, perfluorobutanol, fluorinated alcohols of the formula CF₃(CF₂)ₚ(CH₂CH₂)_{q}—OH where p=1, 2, 3, 4, 5, 6, or 7 and q=0, 1, or 2, and mixtures thereof.

28. The method of embodiment 27, wherein the protic solvent further comprises water and/or an aprotic solvent.

29. The method of embodiment 28, wherein the aprotic solvent used with the protic solvent and/or water is selected from the group consisting of dioxanes, toluene, benzene, para-chlorobenzenetrifluoride, THF, CHCl₃, CH₂Cl₂, perfluorinated alkanes (e.g., perfluorodecalin), tri(fluoroalkyl)amines, and mixtures thereof.

30. The method of any of embodiments 25-29, wherein the mixture is contacted with all or part of the apparatus for 1-25 hours, 1-2 hours, 1-5 hours, 1-10 hours, 1-20 hours, 2-5 hours, 2-10 hours, 2-20 hours, 2-25 hours, 5-10 hours, 5-20 hours, 5-25 hours, 10-20 hours or 10-25 hours.

31. The method of embodiment 30, wherein, following the contacting, the mixture is drained from the apparatus and the apparatus is dried to a substantially constant weight under a stream of gas.

32. The method of embodiment 31, wherein the gas comprises air, nitrogen, oxygen, carbon dioxide, an inert gas (e.g., helium, neon, argon), or a mixture thereof.

33. The method of any of embodiments 13 to 31, wherein, following contacting the anchoring layer molecules with the surface oxygen species to form a covalent bond between the head group of the anchoring layer molecules and the surface, the apparatus is cured at a temperature greater than about 40° C.

34. The method of embodiment 33, wherein the apparatus is cured at a temperature from about 40° C. to about 72° C., about 40° C. to about 60° C., or about 50° C. to about 70° C.

35. The method of embodiment 33 or embodiment 34, wherein the apparatus is cured for about 4 to about 48 hours, about 4 to about 10 hours, about 10 to about 20 hours, about 10 to about 48 hours, about 20 to about 30 hours, about 20 to about 48 hours, about 30 to about 40 hours, or about 30 to about 48 hours.

36. The method of any of embodiments 13 to 35, wherein one or more crosslinking agents are present when contacting the anchoring layer molecules and the surface oxygen species.

37. The method of embodiment 36, wherein the crosslinking agent(s) are selected independently from the group consisting of:
i) one or more independently selected crosslinking agents of the formula (R1O)₃Si—(CH₂)ᵥ—Si(OR1)₃, where v is 0-8 and R1 is alkyl;
ii) one or more independently selected crosslinking agents of the formula (R2)_{4-w}C[(CH₂)ᵥOH]_{w}, where R2 is H, alkyl, OH, or —(CH₂)ₓOH, where w is 2, 3, or 4, x is 1-4, and v is 0-8;
iii) one or more independently selected crosslinking agents of the formula (R4O)₃—Si—R3-Si(OR4), where R3 is —(CF₂)₁₋₆— or —(CH₂)₁₋₆—, and R4 is alkyl or fluoroalkyl (e.g., C1, C2 or C3 alkyl, fluoroalkyl, or perfluoroalkyl);
iv) one or more independently selected crosslinking agents of the formula Cl₃Si—R3-SiCl₃, where R3 is —(CF₂)₁₋₆— or —(CH₂)₁₋₆—; and
v) one or more independently selected crosslinking agents of the formula R5(CH₂CH₂)_{y}Si(OR4)₃, where R4 is alkyl or fluoroalkyl, R5 is —NCO, —NH₂, —NHR4, —N(R4)₂, or epoxy, and y is 1,2,3,or 4.

38. The method of any of embodiments 25-37, wherein the mixture comprising the anchoring layer molecule contacted with the surface comprises from about 1% to about 20%, 1% to 5%, 5% to 10%, or 10% to 20% of the anchoring layer molecules by weight; and optionally comprises from 0% to 5%, 0.1% to 0.2%, 0.2% to 0.5%, 0.5% to 2.0%, or 2.0% to 5.0% of the crosslinker by weight.

39. The method of any preceding embodiment, wherein the lubricating liquid comprises a fluorinated alkane, a fluorinated alkylether, a perfluorinated alkane, a tri(perfluoroalkyl) amine, a perfluoropolyether or a combination thereof.

40. The method of any preceding embodiment, wherein the lubricating liquid comprises a liquid selected from the group consisting of perfluorotripropylamine, perfluorotripentylamine, perfluorotributylamine, perfluorodecalin, perfluoromethyldecalin, perfluorooctane, perfluorobutane, perfluoropropane, perfluoropentane, perfluorohexane, perfluoroheptane, perfluorononane, perfluorodecane, perfluorododecane, perfluorooctyl bromide, perfluoro(2-butyl-tetrahydrofurane), perfluoroperhydrophenanthrene, perfluoroethylcyclohexane, perfluoro(butyltetrahydrofuran), perfluoropolyethers (KRYTOX™, Chemours Co., Wilmington, Del.), 3-ethoxy-1,1,1,2,3,4,4,5,5,6,6,6-dodecafluoro-2-trifluoromethylhexane, trifluoromethane, difluoromethane, pentafluoroethane, or a combination thereof.

41. The method of any of embodiments 1-38, wherein the lubricating liquid comprises butane, pentane, hexane, cyclohexane, heptane, octane, nonane, decane, dodecane, hexadecane, octadecane, triacylglycerides, polydimethyl siloxane, polydiethyl siloxane, fluorinated siloxanes, mineral oil, alkenes, cholesterol, or a combination thereof.

42. The method of any preceding embodiment wherein the apparatus comprises:
a housing having an internal chamber divided into a first part and a second part by one or more membrane(s) (gas exchange membranes), the membranes having a surface exposed to the first part of the chamber and a surface exposed to the second part of the chamber;

wherein the chamber has a liquid inlet and a liquid outlet each of which is in fluid communication with the first part and through which blood (or any other liquid) may enter and exit the first part of the chamber;

wherein the chamber has a gas and/or liquid inlet and a gas and/or liquid outlet each of which is in gas and/or fluid communication with the second part of the chamber and through which a gas (e.g., O₂) or liquid (e.g., to supply O₂ and remove CO₂) may enter and exit the second part of the chamber; and

wherein at least a portion of the surface of the membrane(s) exposed to the first part of the chamber comprises the lubricating liquid surface, the hydrophobic or omniphobic surface and/or the fluid and solid repellant slippery surface.

The first part of the chamber may also be described as the circuit through which blood flows and the second part of the chamber the circuit through which oxygen or a oxygenating medium flow.

43. The method of embodiment 42, wherein at least a portion of the first part of the chamber, in addition to the membrane(s) dividing the chamber into the first and second portions, comprises the lubricating liquid surface, the hydrophobic or omniphobic surface, and/or the fluid and solid repellant slippery surface.

44. The method of embodiment 42 or embodiment 43, wherein at least the first part of the chamber and the second part of the chamber, including the surface of the membrane(s) facing the first part of the chamber, comprise the fluid and solid repellant slippery surface.

45. The method of embodiment 44, wherein the surfaces of the membranes facing the first and second parts of the chamber comprise the lubricating liquid surface, the hydrophobic or omniphobic surface, and/or the fluid and solid repellant slippery surface.

46. The method of any one of embodiments 42-45, wherein the apparatus further comprises:
tubing connected to the liquid inlet and in fluid communication with the first part of the chamber; and/or
tubing connected to the liquid outlet and in fluid communication with the first part of the chamber;

wherein the tubing has an interior surface which comprises a lubricating liquid surface, a hydrophobic or omniphobic surface, and/or a fluid and solid repellant slippery surface.

47. The method of any one of embodiments 42-46, wherein the ECMO apparatus comprises a single membrane separating the first part of the chamber from the second part of the chamber (e.g., a spiral, plaque, laminate or flat sheet ECMO apparatus).

48. The method of any one of embodiments 42-46, wherein the ECMO apparatus is a hollow fiber-type ECMO apparatus with multiple membranes in the form of hollow tubes, wherein the area within the ECMO device chamber around the hollow tubes forms the first part of the chamber and the area within the hollow tubes forms the second part of the chamber.

49. The method of any preceding embodiment, wherein all or part of the ECMO apparatus other than, or in addition to, the gas exchange membrane(s) comprises a polymeric material.

50. The method of embodiment 49, wherein all or part of the apparatus that contacts blood comprises a fluoropolymer and/or perfluoropolymer, including polymers comprising a combination thereof.

51. The method of embodiment 49, wherein the ECMO apparatus comprises one or more polymers selected from FEP, PTFE, PVDF, PFA and silicone, including polymers comprising a combination thereof.

52. The method of any preceding embodiment, wherein all or part of the apparatus that contacts blood comprises a fluoropolymer and/or perfluoropolymer.

53. The method of any of embodiments 46-52, wherein the tubing connected to the liquid inlet and/or tubing connected to the liquid outlet comprises a fluoropolymer and/or perfluoropolymer (including polymers comprising a combination thereof).

54. The method of embodiment 53, wherein the tubing connected to the liquid inlet and/or tubing connected to the liquid outlet is lined with a fluoropolymer and/or perfluoropolymer (including polymers comprising a combination thereof).

55. The method of any of embodiments 53-54, wherein the tubing connected to the liquid inlet and/or tubing connected to the liquid outlet comprises polyurethane, PVC or silicone tubing lined with FEP, PTFE, PFA, and/or polymers comprising a combination thereof.

56. The method of any preceding embodiment wherein the ECMO apparatus further comprises a pump (e.g., a centrifuge, peristaltic or diaphragm pump), wherein all or part of the pump that contacts blood comprises a surface coated with a lubricating liquid, a fluid and solid repellant slippery surface, and/or a hydrophobic or omniphobic surface.

57. The method of embodiment 56, wherein all or part of the pump that contacts blood and comprises the surface coated with the lubricating liquid, the fluid and solid repellant slippery surface, and/or the hydrophobic or omniphobic surface, is a pump part comprised of or lined with a fluoropolymer and/or perfluoropolymer.

58. The method of embodiment 57, wherein the fluoropolymer and/or perfluoropolymer is selected from FEP, PTFE, PFA, and/or polymers comprising a combination thereof.

59. An ECMO apparatus (e.g., an apparatus comprising an ECMO oxygenator) prepared by the method of any preceding embodiment.

60. An ECMO apparatus (e.g., an apparatus comprising an ECMO oxygenator) prepared by the method of any of embodiments 1-58 for use in a method of supplying oxygen to and/or removing carbon dioxide from blood (e.g., blood from a patient or a perfused organ).

61. An ECMO apparatus of embodiment 59 or embodiment 60, comprising lubricating liquid surface(s) on all or part of the apparatus that contacts blood (e.g., the gas exchange membrane(s), housing, tubing, and/or pump).

62. The ECMO apparatus of embodiment 61, wherein the lubricating liquid surface is formed on a fluoropolymer and/or perfluoropolymer surface of the apparatus using a fluorinated and/or perfluorinated lubricating liquid that becomes substantially immobilized thereon.

63. The ECMO apparatus of embodiment 62, wherein the lubricating liquid surface is formed on a fluoropolymer and/or perfluoropolymer surface of the apparatus using perfluorinated lubricating liquid that becomes substantially immobilized thereon (to form a FILP surface).

64. An ECMO apparatus of embodiment 59 or embodiment 60, comprising a fluid and solid repellant slippery surface(s) on all or part of the apparatus that contacts blood (e.g., the gas exchange membrane(s), housing, tubing, and/or pump).

65. The ECMO apparatus of embodiment 64, wherein the fluid and solid repellant slippery surface is formed on a fluoropolymer and/or perfluoropolymer surface of the apparatus using a fluorinated and/or perfluorinated lubricating liquid that becomes substantially immobilized thereon.

66. The ECMO apparatus of embodiment 65, wherein the fluid and solid repellant slippery surface is formed on a fluoropolymer and/or perfluoropolymer surface of the apparatus using perfluorinated lubricating liquid that becomes substantially immobilized thereon.

67. The ECMO apparatus of any of embodiments 61-66, wherein the lubricating liquid surface and/or the fluid and solid repellant slippery surface(s) of the apparatus resist the colonization and/or biofilm formation by bacteria (e.g., Staphylococcus epidermidis strain ATCC 14990) or fungi (e.g., Candida).

68. The ECMO apparatus of embodiment 67, wherein the lubricating liquid surface and/or the fluid and solid repellant slippery surface(s) of the apparatus resist colonization and/or biofilm formation by bacteria (e.g., Staphylococcus epidermidis strain ATCC 14990).

69. The ECMO apparatus of embodiment 68, wherein the resistance to colonization and/or biofilm formation of a sample of the lubricating liquid surface and/or the fluid and solid repellant slippery surface(s) is based on Flow Loop Durability relative to an otherwise identical control sample that has not been contacted with a lubricating liquid (e.g., a fluorinated or perfluorinated liquid such as a fluorocarbon, perfluorocarbon, tri(fluoroalkyl)amine, and combinations thereof).

70. The ECMO apparatus of embodiment 69, wherein the resistance to colonization and/or biofilm formation is a greater than 70%, greater than 80%, greater than 85%, or greater than 90% reduction in bacteria (e.g., Staphylococcus epidermidis strain ATCC 14990) or fungi (e.g., Candida) associated with the lubricating liquid surface and/or the fluid and solid repellant slippery surface(s) after Flow Loop Durability testing in which the sample surface and the control sample are subjected to flowing phosphate buffered saline for up to 60 days (e.g., up to 10 days, 20 days, 30 days, 40 days, or 50 days) prior to challenge by the bacteria or fungi.

71. The ECMO apparatus of any of embodiments 6170, wherein the lubricating liquid is substantially immobilized on the lubricating liquid surface(s) and/or the fluid and solid repellant slippery surface(s), such that a sample surface (a sample lubricating liquid surface or sample fluid and solid repellant slippery surface) displays a reduction of greater than 70%, (e.g., greater than 80%, greater than 85%, or greater than 90%) in associated Staphylococcus epidermidis strain ATCC 14990 relative to an otherwise identical control sample that has not been contacted with a lubricating liquid (e.g., a fluorinated or perfluorinated liquid such as a fluorocarbon or perfluorocarbon liquid) after Flow Loop Durability testing in which the sample surface and the control sample are subjected to flowing phosphate buffered saline for up to 60 days (e.g., up to 10 days, 20 days, 30 days, 40 days, or 50 days) prior to challenge by Staphylococcus epidermidis strain ATCC 14990. (In other words, the lubricating liquid is substantially immobilized (bound tightly enough) such that subjecting the sample surface to flowing saline does not remove the lubricating liquid as measured by its continued ability to resist bacterial colonization or biofilm growth.)

72. An ECMO apparatus (e.g., an oxygenator) of any of embodiments 62-71 for use in a method of supplying oxygen to and/or removing carbon dioxide from blood (e.g., blood from a patient or a perfused organ).

73. A method of supplying oxygen to and/or removing carbon dioxide from blood comprising: passing blood through the first part of the chamber of an ECMO oxygenator apparatus of any of embodiments 59-72 and an oxygen, or an oxygen contain gas or liquid, through the second part of the chamber of the apparatus.

74. The method of embodiment 73, wherein the blood is removed from a living subject prior to supplying oxygen to and/or removing carbon dioxide from the blood and returned to the living subject thereafter.

75. The method of embodiment 73, wherein the blood is removed from a perfused organ or perfused organs prior to supplying oxygen to and/or removing carbon dioxide from the blood and returned to the perfused organ or perfused organs thereafter.

76. An ECMO pump (e.g., a centrifuge, peristaltic or diaphragm pump) wherein one or more components of the pump that contact blood comprise lubricating liquid surface(s) and/or a fluid and solid repellant slippery surface(s) on all or part of the one or more components that come into contact with blood.

77. The ECMO pump of embodiment 76, wherein the lubricating liquid surface(s) and/or the fluid and solid repellant slippery surface(s) are formed on a fluoropolymer and/or perfluoropolymer surface of the component using a fluorinated and/or perfluorinated lubricating liquid that becomes substantially immobilized thereon.

78. The ECMO pump of embodiment 77, wherein the fluoropolymer and/or perfluoropolymer surface is a coating applied to all or part of one or more of the pump components.

79. The ECMO pump of embodiment 77 or embodiment 78, wherein the fluoropolymer and/or perfluoropolymer comprises FEP, PTFE, PFA, or a combination thereof.

80. Tubing for use in blood transport (e.g., attached to a membrane oxygenator of an ECMO apparatus) comprising a lubricating liquid surface(s) and/or a fluid and solid repellant slippery surface(s) on all or part of the tubing (e.g., the inner and/or outer surface of the tubing) that contacts blood.

81. The tubing of embodiment 80, wherein the lubricating liquid surface(s) and/or the fluid and solid repellant slippery surface(s) are formed on a fluoropolymer and/or perfluoropolymer surface of the tubing using a fluorinated and/or perfluorinated lubricating liquid that becomes substantially immobilized thereon.

82. The tubing of embodiment 81, wherein the fluoropolymer and/or perfluoropolymer comprises FEP, PTFE, PFA, or a combination thereof.

83. The tubing of embodiment 81 or 82, wherein the fluoropolymer and/or perfluoropolymer surface is a coating (e.g., from about 0.5 to 5 microns thick) on the inner and/or outer surface of the tubing.

84. The tubing of embodiments 83, wherein the tubing is formed from PVC or silicone and lined with the fluoropolymer and/or perfluoropolymer.

85. The tubing of embodiment 84, wherein the tubing is lined with FEP or PTFE.

86. An extracorporeal membrane oxygenator (“ECMO”) oxygenator apparatus, the oxygenator comprising:
a housing having an internal chamber divided into a first part and a second part by one or more membrane(s), the membranes having a surface exposed to the first part of the chamber and a surface exposed to the second part of the chamber;

wherein the chamber has a liquid inlet and a liquid outlet each of which is in fluid communication with the first part (e.g., through which blood may be introduce into and leave the first part of the chamber);

wherein the chamber has a gas or liquid inlet and a gas or liquid outlet each of which is in gas and/or fluid communication with the second part of the chamber; and

wherein at least a portion of the surface of the membrane(s) exposed to the first part of the chamber comprises a lubricating liquid surface and/or a fluid and solid repellant slippery surface.

87. The oxygenator apparatus of embodiment 86, wherein the membrane comprises one or more polymers.

88. The oxygenator apparatus of embodiment 86 or embodiment 87, wherein the membrane comprises one or more of polyolefins, silicones, fluoropolymers and/or perfluoropolymers.

89. The oxygenator apparatus of any of embodiments 86-88, wherein the membrane comprises one or more of poly-4-methyl pentene, propylene, FEP (fluorinated ethylene propylene), PTFE (polytetrafluoroethylene), PVDF (polyvinylidene fluoride), silicone, PFA (perfluoroalkoxy alkane(s), and combinations thereof.

90. The oxygenator apparatus of embodiment 89, wherein the membrane comprises poly-4-methyl pentene and/or FEP.

91. The oxygenator apparatus of any of embodiments 86-90, comprising a fluid and solid repellant slippery surface on all or part the membrane.

92. The oxygenator apparatus of embodiment 91, wherein:

the membrane comprises poly-4-methyl pentene, propylene, FEP (fluorinated ethylene propylene), PTFE (polytetrafluoroethylene), PVDF (polyvinylidene fluoride), silicone, PFA (perfluoroalkoxy alkane(s), and combinations thereof; and

the fluid and solid repellant slippery surface comprises one or more anchoring molecules comprising fluoroalkyl and/or perfluoroalkyl groups.

93. The oxygenator apparatus of embodiment 92, where the membrane comprises poly-4-methyl pentene and/or FEP, and the anchoring molecules comprise fluoroalkyl and/or perfluoroalkyl groups having from about 6 to about 16 carbon atoms.

94. The oxygenator apparatus of any of embodiments 86-93, wherein the lubricating liquid surface and/or the fluid and solid repellant slippery surface comprises a fluorinated or perfluorinated lubricating liquid.

95. The oxygenator apparatus of any of embodiments 88-90, wherein the lubricating liquid surface and/or the fluid and solid repellant slippery surface comprises a fluorinated or perfluorinated lubricating liquid.

96. The oxygenator apparatus of any of embodiments 86-95, wherein the lubricating liquid comprises a fluorinated alkane, a fluorinated alkylether, a perfluorinated alkane, a tri(perfluoroalkyl) amine, a perfluoropolyether or a combination thereof.

97. The oxygenator apparatus of embodiment 96, wherein the lubricating liquid comprises a liquid selected from the group consisting of perfluorotripropylamine, perfluorotripentylamine, perfluorotributylamine, perfluorodecalin, perfluoromethyldecalin, perfluorooctane, perfluorobutane, perfluoropropane, perfluoropentane, perfluorohexane, perfluoroheptane, perfluorononane, perfluorodecane, perfluorododecane, perfluorooctyl bromide, perfluoro(2-butyl-tetrahydrofurane), perfluoroperhydrophenanthrene, perfluoroethylcyclohexane, perfluoro(butyltetrahydrofuran), perfluoropolyethers (KRYTOX™), 3-ethoxy-1,1,1,2,3,4,4,5,5,6,6,6-dodecafluoro-2-trifluoromethylhexane, trifluoromethane, difluoromethane, pentafluoroethane, or a combination thereof.

98. The oxygenator apparatus of any of embodiments 86-97, wherein the membrane(s) comprises (i) the lubricating liquid surface formed on a fluoropolymer and/or perfluoropolymer and/or (ii) a fluid and solid repellant slippery surface, and wherein the fluorinated and/or perfluorinated lubricating liquid becomes substantially immobilized to the lubricating liquid surface or the fluid and solid repellant slippery surface such that after Flow Loop Durability testing in which a sample surface and a control sample are subjected to flowing phosphate buffered saline (50 mL/min in a 6.3 mm inner diameter tube at 37° C.) for up to 60 days, the sample lubricating liquid surface or sample fluid and solid repellant slippery surface displays a reduction of greater than 70% in associated Staphylococcus epidermidis strain ATCC 14990 relative to the otherwise identical control sample that has not been contacted with a lubricating liquid when challenged by placement in a growing culture of Staphylococcus epidermidis strain ATCC 14990 for 48 hours.

99. The oxygenator apparatus of any of embodiments 86-97, wherein the membrane(s) comprises (i) the lubricating liquid surface formed on a fluoropolymer and/or perfluoropolymer and/or (ii) a fluid and solid repellant slippery surface, and wherein the lubricating liquid surface and/or the fluid and solid repellant slippery surface display a reduction of greater than 70% in associated Staphylococcus epidermidis strain ATCC 14990 relative to an otherwise identical control sample that has not been contacted with a lubricating liquid when challenged by placement in a growing culture of Staphylococcus epidermidis strain ATCC 14990 for 48 hours after Flow Loop Durability testing in which the sample surface and the control sample are subjected to flowing phosphate buffered saline (50 mL/min in a 6.3 mm inner diameter tube at 37° C.) for up to 60 days.

100. The ECMO oxygenator apparatus of any of embodiments 86-99, further comprising one or more pumps, reservoirs, and/or tubing associated with the oxygenator to form an ECMO apparatus.

101. The ECMO apparatus of embodiment 100, wherein at least one surface of the one or more pumps, reservoirs, and/or tubing comprises a lubricating liquid surface, a hydrophobic and/or omniphobic surface or a fluid and solid repellant slippery surface.

102. The ECMO apparatus of embodiment 100 or embodiment 101, wherein at least one surface of the one or more pumps, reservoirs, and/or tubing comprises the lubricating liquid surface, formed on a fluoropolymer and/or perfluoropolymer, and/or a fluid and solid repellant slippery surface.

103. The ECMO apparatus of any of embodiments 100-102, wherein the lubricating liquid surface and/or a fluid and solid repellant slippery surface on one or more pumps, reservoirs, and/or tubing comprises fluorinated and/or perfluorinated lubricating liquids.

104. The oxygenator apparatus of any of embodiments 86-103 for use in a method of supplying oxygen to and/or removing carbon dioxide from blood.

105. An ECMO pump wherein one or more components of the pump that contact blood comprise lubricating liquid surface(s) and/or fluid and solid repellant slippery surface(s) on all or part of the surfaces of the component(s) that contact blood.

106. The ECMO pump of embodiment 105, wherein the lubricating liquid surface(s) and/or fluid and solid repellant slippery surface(s) comprise one or more fluorinated and/or perfluorinated lubricating liquids that becomes substantially immobilized thereon; and wherein, when the lubricating liquid surface is present, it is formed on a fluoropolymer and/or perfluoropolymer surface of the component.

107. The ECMO pump of embodiment 106, wherein the fluoropolymer and/or perfluoropolymer surface is a coating applied to all or part of one or more of the pump components.

108. The ECMO pump of embodiment 106 or embodiment 107, wherein the fluoropolymer and/or perfluoropolymer comprises FEP, PTFE, PFA, or a combination thereof.

109. Tubing having an inner and/or outer surface formed by a coating or liner, wherein the tubing comprises a lubricated liquid surface and/or a fluid and solid repellant slippery surface on all or part of the inner and/or outer surface of the tubing, wherein the lubricating liquid surface(s) and/or fluid and solid repellant slippery surface(s) comprise one or more fluorinated and/or perfluorinated lubricating liquids that become substantially immobilized thereon; and wherein when the lubricating liquid surface is present, it is formed on a fluoropolymer and/or perfluoropolymer surface of the coating or liner.

110. The tubing of embodiment 109, wherein the tubing is a silicone, PVC, or polyurethane tubing having a fluoropolymer and/or perfluoropolymer coating or liner.

111. The tubing of embodiment 109 or 110, wherein the fluoropolymer and/or perfluoropolymer comprises FEP, PTFE, PFA, or a combination thereof.

112. A method of conducting ECMO on the blood of a patient/subject, the method comprising: passing blood from the patient/subject through the oxygenator of an ECMO apparatus of embodiments 59-72, while providing an oxygen supply medium (e.g., gaseous oxygen or an oxygenated liquid) to the oxygenator, thereby providing oxygen to the blood and/or removing carbon dioxide from the blood, and returning the blood to the patient/subject; wherein the blood and the oxygen supply medium are separated by the oxygenator's gas exchange membrane(s).

113. A method of conducting ECMO on the blood of a patient/subject, the method comprising: passing blood from the patient/subject through the first part of an oxygenator of an ECMO apparatus of embodiments 86-104 while providing an oxygen supply medium (e.g., gaseous oxygen or an oxygenated liquid) to the second part of the oxygenator, thereby providing oxygen to the blood and/or removing carbon dioxide from the blood, and returning the blood to the patient/subject; wherein the blood and the oxygen supply medium are separated by the oxygenator's gas exchange membrane(s).

### EXAMPLE

Cleaning: An ECMO device is cleaned of any material that might interfere with the activation of the surface for reaction with anchoring layer molecules and/or crosslinkers that may be used with the anchoring layer molecules. Cleaning is accomplished by washing with phosphate-buffered saline (PBS, 10 mM phosphate pH 7.4, 137 mM NaCl, 2.7 mM KCl) containing 5% SDS to remove, for example, any protein (e.g., BSA)/heparin coating on the ECMO apparatus. The PBS buffer with 5% SDS is circulated through the ECMO circuit including the pump and oxygenator (the housing including the gas exchange membrane) overnight. Distilled water is used to rinse the PBS/SDS from the apparatus, and the circuit is dried with constant air flow until a steady weight is attained. ECMO devices that are not coated with BSA/heparin do not necessarily require cleaning with PBS/SDS but it may be conducted to remove other trace contaminants.

Surface activation: Following cleaning as described above, the ECMO circuit, oxygenator and catheters are individually activated using O₂ plasma at 300-400 mTorr oxygen pressure or under O₂ plasma activation conditions which yields a water contact angle of 60°-10° (e.g., 60°-40 °, 40°-30°, 30°-20° or 20°-10°) on PVC surfaces and 70°-10° (e.g., 70°-60°, 60°-50°, 50°-40°, 40°-30°, 30°-20°, 20°-10° or 10°-0°) on poly-4-methyl pentene surfaces.

Treatment with anchoring layer molecules and crosslinkers: Immediately after surface activation with oxygen plasma, the entire circuit is exposed to (contacted with) a reaction mixture containing at least one anchoring layer molecule and any crosslinker that may be utilized. The reaction mixture is circulated through the ECMO device for 20 hours after which it is drained from the device. The device is dried utilizing a constant flow of air or another gas (e.g., nitrogen) until a constant weight is obtained. The treated device is cured at 66° C. (150° F.) for 48 hours. Unreacted anchoring layer molecules and crosslinkers are removed by rinsing with a solvent in which they are soluble (e.g., perfluorodecalin) and the circuit is dried using a stream of air or another gas (e.g., nitrogen).

Reaction Mixtures Depending on the reactivity of the crosslinking reagents and anchoring layer molecule head groups, different reaction mixtures incorporating those materials may be desirable. Where either or both of the crosslinker and head group react with protic solvents (e.g., they contain trichloro silane groups), an aprotic solvent mixture may be employed. In contrast, a reaction utilizing protic solvent may be utilized where either or both of the crosslinker and the head group of the anchoring layer molecules are stable in protic solvents. Protic solvents may also be utilized when it is desirable to prehydrolyze/pre-react certain reactive groups in the crosslinkers or anchoring groups to form species compatible with the protic solvent (e.g., reacting a trichlorosilane with a protic solvent such as methanol to form a trimethoxy silane, which may react with hydroxl groups on the ECMO apparatus surface and is compatible with the methanol as a solvent.

a. Aprotic Solvent Reaction Mixture: By way of example an aprotic reaction mixture is formed by combining an anchoring layer molecule such as (tridecafluoro-1,1,2,2-tetrahydrooctyl)trichlorosilane and any desired crosslinking agent in an aprotic solvent or a mixture of aprotic solvents. Some suitable aprotic solvents include dioxanes, toluene, benzene, para-chlorobenzenetrifluoride, THF, CHCl₃, CH₂Cl₂, or a perfluorinated solvent (e.g., perfluorodecalin, or FLUORINERT™ (e.g., FC 43, FC70 or FC 72, 3M Company). The reaction mixture may be applied to all or part of the ECMO apparatus to modify the surface by covalently binding the anchoring layer molecules to the surface. Where groups such as trichlorosilane functionalities are present in the crosslinker or anchoring layer molecules, care to vent the HCl gas must be taken. The HCl generated may also be damaging to the apparatus components including the membrane used for carbon dioxide and oxygen exchange. In such reaction mixtures, the anchoring layer molecule and crosslinker together comprise up to 25% of the reaction mixture by weight with the remainder of the composition comprised of the aprotic solvents and any catalyst employed. In some embodiments the anchoring layer molecule is present in the reaction mixture in an amount from about 1% to about 20% (e.g., 1% to 5%, 5% to 10%, or 10% to 20%) of the reaction mixture by weight and from 0 to about 5% (e.g., 0.1% to 0.2%, 0.2% to 0.5%, 0.5% to 2.0% or 2.0% to 5.0%) crosslinker by weight with the remainder of the composition comprised of the aprotic solvents and any catalyst employed.

b. Protic Reaction Mixture: By way of example, a reaction mixture employing protic solvent(s) may be formed by combining an anchoring layer molecule such as tridecafluoro-1,1,2,2-tetrahydrooctyl)triethoxysilane and any desired crosslinking agent and a protic solvent. Some suitable protic solvents include methanol, ethanol, isopropanol, butanol, perfluorobutanol, fluorinated alcohols such as CF₃(CF₂)ₚ(CH₂CH₂)_{q}—OH where p=1, 2, 3, 4, 5, 6, or 7 and q=0, 1, or 2 or water. Aprotic solvent may also be present in protic solvent reaction mixtures e.g., dioxanes, toluene, benzene, para-chlorobenzenetrifluoride, THF, CHCl₃, CH₂Cl₂, or perfluorinated solvent (e.g. perfluorodecalin or FLUORINERT™ FC 43, FC70 or FC 72). When trichlorosilanes such as (tridecafluoro-1,1,2,2-tetrahydrooctyl)trichlorosilane are utilized with protic solvents such as alcohols, alkoxy silanes are generated along with HCl. The HCl can serve to hydrolyzes the alkoxy silane to produce reactive silanols or silanol oligomers in situ. In some embodiments the anchoring layer molecule is present in the reaction mixture in an amount from about 1% to about 20% (e.g., 1% to 5%, 5% to 10%, or 10% to 20%) of the reaction mixture by weight, and the crosslinker comprises from 0 to about 5% (e.g., 0.1% to 0.2%, 0.2% to 0.5%, 0.5% to 2.0% or 2.0% to 5.0%) of the reaction mixture by weight with the remainder of the composition comprised of the aprotic solvents and any catalyst employed. In some embodiments where water and/or aprotic solvents are employed they may make up less than about 80% (e.g., less than 70%, 60%, 50%, 40%, 30%, 20%, 10%, 5%, or 1%) of the reaction mixture by weight, provided the reaction mixture comprises at least 1% of a protic solvent by weight.

The following table summarizes some reaction mixtures for the surface treatment of ECMO devices following activation with oxygen plasma.

| | | | | | Pre-hydrolysis |
|---|---|---|---|---|---|
| Condition | Silane | Crosslinker | Solvent | Catalyst | conditions |
| A | (Tridecafluoro-1,1,2,2- | No | Perfluorodecalin | No | No |
| | tetrahydrooctyl) | | | | |
| | trichlorosilane (5%) | | | | |
| B | (Tridecafluoro-1,1,2,2- | Tetraethoxysilane | Methanol:water | Acetic acid | Yes, 20 hours |
| | tetrahydrooctyl)triethoxy- | (0.2%) | (80:20) | | room |
| | silane(4.8%) | | | | temperature |
| C | (Tridecafluoro-1,1,2,2- | No | Water in methanol | Acetic | Yes, 20 hours |
| | tetrahydrooctyl)triethoxy- | | | acid/metal | room |
| | silane (5%) | | | catalyst | temperature |
| D | (Tridecafluoro-1,1,2,2- | tetraethoxysilane | protic solvent | Acetic | Yes, 20 hours |
| | tetrahydrooctyl)triethoxy- | | containing water | acid/metal | room |
| | silane | | | catalyst | temperature |
| E | (Tridecafluoro-1,1,2,2- | (R1O)₃Si—(CH₂)ᵥ—Si(OR1)₃ | protic solvent | Acetic | Yes, 20 hours |
| | tetrahydrooctyl)triethoxy- | where v is 1-8, and R1 is alkyl | containing water | acid/metal | room |
| | silane | (e.g., methyl or ethyl) | | catalyst | temperature |
| F | (Tridecafluoro-1,1,2,2- | (R2)_{4-w}C[(CH₂)ᵥOH]_{w} where | Protic solvent | Acetic | Yes, 20 hours |
| | tetrahydrooctyl)triethoxy- | R2 is H, alkyl, OH, or —(CH₂)ₓOH | containing water | acid/metal | room |
| | silane | w is 2, 3, or 4, | | catalyst | temperature |
| | | x is 1-4, and | | | |
| | | v is 1-8 | | | |
| G | (Tridecafluoro-1,1,2,2- | (R4O)₃₋Si—R3—Si(OR4), where R3 | protic solvent | Acetic | Yes, 20 hours |
| | tetrahydrooctyl)triethoxy- | is —(CF₂)₁₋₆— or —(CH₂)₁₋₆—, and R4 | containing water | acid/metal | room |
| | silane | is alkyl or fluoroalkyl | | catalyst | temperature |
| H | (Tridecafluoro-1,1,2,2- | Cl₃Si—R3—SiCl₃, where R3 | Aprotic solvent | None | No |
| | tetrahydrooctyl)trichloro- | is —(CF₂)₁₋₆— or —(CH₂)₁₋₆— | | | |
| | silane | | | | |
| I | (Tridecafluoro-1,1,2,2- | None | Aprotic solvent | None | No |
| | tetrahydrooctyl) | | | | |
| | trichlorosilane | | | | |
| J | (Tridecafluoro-1,1,2,2- | R5(CH₂CH₂)_{y}Si(OR4)₃ | protic solvent | Acetic | No |
| | tetrahydrooctyl)triethoxy- | where | containing water | acid/metal | |
| | silane | R4 is alkyl or fluoroalkyl (e.g., | | catalyst | |
| | | methyl or ethyl), | | | |
| | | R5 is —NCO, —NH₂, —NHR4, | | | |
| | | —N(R4)₂ or epoxy, and | | | |
| | | y is 1, 2, 3, or 4 | | | |

1. An extracorporeal membrane oxygenator (“ECMO”) oxygenator apparatus, the oxygenator comprising:
a housing having an internal chamber divided into a first part and a second part by one or more membrane(s), the membranes having a surface exposed to the first part of the chamber and a surface exposed to the second part of the chamber;
wherein the chamber has a liquid inlet and a liquid outlet each of which is in fluid communication with the first part;
wherein the chamber has a gas or liquid inlet and a gas or liquid outlet each of which is in gas and/or fluid communication with the second part of the chamber; and
wherein at least a portion of the surface of the membrane(s) exposed to the first part of the chamber comprises a lubricating liquid surface and/or a fluid and solid repellant slippery surface.

2. The apparatus of claim 1, wherein the membrane(s) comprises one or more polymers.

3. The apparatus of claim 2, wherein the membrane(s) comprises one or more of polyolefin, silicone, fluoropolymer and/or perfluoropolymer.

4. The apparatus of claim 3, wherein the membrane(s) comprises one or more of poly-4-methyl pentene, propylene, FEP (fluorinated ethylene propylene), PTFE (polytetrafluoroethylene), PVDF (polyvinylidene fluoride), silicone, PFA (perfluoroalkoxy alkane(s), and combinations thereof.

5. The apparatus of claim 3, wherein the membrane(s) comprises poly-4-methyl pentene and/or FEP.

6. The apparatus of claim 3, comprising a lubricating liquid surface on all or part of the membrane(s).

7. The apparatus of claim 6, wherein the membrane(s) comprises one or more of poly-4-methyl pentene, propylene, FEP (fluorinated ethylene propylene), PTFE (polytetrafluoroethylene), PVDF (polyvinylidene fluoride), silicone, PFA (perfluoroalkoxy alkane(s), and combinations thereof.

8. The apparatus of claim 7, wherein the membrane(s) comprises poly-4-methyl pentene and/or FEP.

9. The apparatus of claim 3, comprising a fluid and solid repellant slippery surface on all or part the membrane(s).

10. The apparatus of claim 9, wherein:
the membrane(s) comprises one or more of poly-4-methyl pentene, propylene, FEP (fluorinated ethylene propylene), PTFE (polytetrafluoroethylene), PVDF (polyvinylidene fluoride), silicone, PFA (perfluoroalkoxy alkane(s), and combinations thereof; and
the fluid and solid repellant slippery surface comprises one or more anchoring molecules comprising fluoroalkyl and/or perfluoroalkyl groups.

11. The apparatus of claim 10, wherein the membrane(s) comprises poly-4-methyl pentene and/or FEP.

12. The apparatus of claim 10, wherein the membrane comprises poly-4-methyl pentene and/or FEP and the anchoring molecules comprise fluoroalkyl and/or perfluoroalkyl groups having from about 6 to about 16 carbon atoms.

13. The apparatus of claim 1, wherein the lubricating liquid surface and/or the fluid and solid repellant slippery surface comprise a fluorinated or perfluorinated lubricating liquid.

14. The apparatus of claim 13 wherein the lubricating liquid comprises a fluorinated alkane, a fluorinated alkylether, a perfluorinated alkane, a tri(perfluoroalkyl) amine, a perfluoropolyether or a combination thereof.

15. The apparatus of claim 13, wherein the lubricating liquid comprises a liquid selected from the group consisting of perfluorotripropylamine, perfluorotripentylamine, perfluorotributylamine, perfluorodecalin, perfluoromethyldecalin, perfluorooctane, perfluorobutane, perfluoropropane, perfluoropentane, perfluorohexane, perfluoroheptane, perfluorononane, perfluorodecane, perfluorododecane, perfluorooctyl bromide, perfluoro(2-butyl-tetrahydrofurane), perfluoroperhydrophenanthrene, perfluoroethylcyclohexane, perfluoro(butyltetrahydrofuran), perfluoropolyethers (KRYTOX™), 3-ethoxy-1,1,1,2,3,4,4,5,5,6,6,6-dodecafluoro-2-trifluoromethylhexane, trifluoromethane, difluoromethane, pentafluoroethane, or a combination thereof.

16. The apparatus of claim 13, wherein the membrane(s) comprises (i) the lubricating liquid surface formed on a fluoropolymer and/or perfluoropolymer and/or (ii) a fluid and solid repellant slippery surface, wherein the fluorinated and/or perfluorinated lubricating liquid becomes substantially immobilized to the lubricating liquid surface or the fluid and solid repellant slippery surface such that after Flow Loop Durability testing in which a sample surface and a control sample are subjected to flowing phosphate buffered saline (50 mL/min in a 6.3 mm inner diameter tube) for up to 60 days, a sample lubricating liquid surface or sample fluid and solid repellant slippery surface displays a reduction of greater than 70% in associated Staphylococcus epidermidis strain ATCC 14990 relative to an otherwise identical control sample that has not been contacted with a lubricating liquid when challenged by placement in a growing culture of Staphylococcus epidermidis strain ATCC 14990 for 48 hours.

17. The oxygenator apparatus of claim 13, wherein the membrane(s) comprises (i) the lubricating liquid surface formed on a fluoropolymer and/or perfluoropolymer and/or (ii) a fluid and solid repellant slippery surface, and wherein the lubricating liquid surface and/or the fluid and solid repellant slippery surface display a reduction of greater than 70% in associated Staphylococcus epidermidis strain ATCC 14990 relative to an otherwise identical control sample that has not been contacted with a lubricating liquid when challenged by placement in a growing culture of Staphylococcus epidermidis strain ATCC 14990 for 48 hours after Flow Loop Durability testing in which the sample surface and the control sample are subjected to flowing phosphate buffered saline (50 mL/min in a 6.3 mm inner diameter tube at 37° C.) for up to 60 days.

18. The oxygenator apparatus of claim 13, further comprising one or more pumps, reservoirs, and/or tubing associated with the oxygenator to form an ECMO apparatus.

19. The apparatus of claim 18, wherein at least one surface of the one or more pumps, reservoirs, and/or tubing comprises a lubricating liquid surface, a hydrophobic and/or omniphobic surface or a fluid and solid repellant slippery surface.

20. The apparatus of claim 19, wherein at least one surface of the one or more pumps, reservoirs, and/or tubing comprises the lubricating liquid surface formed on a fluoropolymer and/or perfluoropolymer, and/or a fluid and solid repellant slippery surface.

21. The apparatus of claim 20, wherein the lubricating liquid surface and/or a fluid and solid repellant slippery surface on one or more pumps, reservoirs, and/or tubing comprise fluorinated and/or perfluorinated lubricating liquids.

22. The apparatus of claim 13 for use in a method of supplying oxygen to and/or removing carbon dioxide from blood.

23. The apparatus of claim 21 for use in a method of supplying oxygen to and/or removing carbon dioxide from blood.

24. An ECMO pump wherein one or more components of the pump that contact blood comprises lubricating liquid surface(s) and/or fluid and solid repellant slippery surface(s) on all or part of the surfaces of the component(s) that contact blood.

25. The ECMO pump of claim 24, wherein the lubricating liquid surface(s) and/or fluid and solid repellant slippery surface(s) comprise one or more fluorinated and/or perfluorinated lubricating liquids that become substantially immobilized thereon; and wherein, when the lubricating liquid surface is present, it is formed on a fluoropolymer and/or perfluoropolymer surface of the component.

26. Tubing having an inner and/or outer surface formed by a coating or liner, wherein the tubing comprises a lubricated liquid surface and/or a fluid and solid repellant slippery surface on all or part of the inner and/or outer surface of the tubing, wherein the lubricating liquid surface(s) and/or fluid and solid repellant slippery surface(s) comprise one or more fluorinated and/or perfluorinated lubricating liquids that become substantially immobilized thereon; and wherein, when the lubricating liquid surface is present, it is formed on a fluoropolymer and/or perfluoropolymer surface of the coating or liner.

27. The tubing of claim 26, wherein the tubing is a silicone, PVC, or polyurethane tubing having a fluoropolymer and/or perfluoropolymer coating or liner.

28. The tubing of claim 26, wherein the fluoropolymer and/or perfluoropolymer comprises FEP, PTFE, PFA, or a combination thereof.
